(19) United States
(12) Patent Application Publication
(10) Pub. No.: US 2005/0036971 A1
(43) Pub. Date: Feb. 17, 2005

(54) Pearlescent cosmetic or dermatological formulations

(75) Inventors: Silke Kohlhase, Hamburg (DE); Andreas Bleckmann, Ahrensburg (DE); Heidi Riedel, Hamburg (DE); Stefanie Von Thaden, Hamburg (DE)

Correspondence Address: GREENBLUM & BERNSTEIN, P.L.C., 1950 ROLAND CLARKE PLACE, RESTON, VA, 20191, US

(73) Assignee: BEIERSDORF AG, Hamburg (DE)

(21) Appl. No.: 10/759,160

(22) Filed: Jan. 20, 2004

(30) Foreign Application Priority Data
Jan. 20, 2003 (DE) 103 01 836.0

(51) Int. Cl. A61K 7/06; A61K 7/11; A61K 7/075; A61K 7/08

(52) U.S. Cl. 424/70.11; 424/70.22; 424/70.12; 424/70.16

(57) ABSTRACT

A cosmetic or dermatological preparation which comprises a C₁₂-C₄₀ fatty acid, a C₁₂-C₄₀ fatty alcohol, an amphiphilic polymer, an associative polymer and/or a siloxane elastomer, and sodium and/or potassium hydroxide.

The invention relates to cosmetic preparations with a pearlescent effect and good skin compatibility, and to the use thereof.

Customary cosmetic application forms and preparations are often emulsions. This term is generally understood as meaning a heterogeneous system of two liquids which are immiscible or miscible only to a limited extent with one another and are usually referred to as phases. One is in the form of droplets (disperse or internal phase), whilst the other liquid forms a continuous (coherent or internal) phase. Less common application forms are multiple emulsions, i.e. those which, in the droplets of the dispersed (or discontinuous) phase, comprise for their part droplets of a further dispersed phase, e.g. W/O/W emulsions and O/W/O emulsions.

More recent findings have recently led to a better understanding of cosmetic emulsions which are of relevance in practice. Here, it is assumed that the emulsifier mixtures used in excess form lamellar liquid-crystalline phases or crystalline gel phases. In the gel network theory, stability and physicochemical properties of such emulsions are attributed to the formation of viscoelastic gel networks. In order to be able to ensure the metastability of emulsions, surface-active substances, i.e. emulsifiers, are usually necessary.

Emulsions from the prior art have a pearlescent effect, as is described, for example, in WO 0110403, WO 9010429, DE 19921186 or DE 19944545. These cosmetic preparations comprise mono- and di-fatty acid esters of glycerol or glycol, such as, for example glycerol stearates, laurates or myristates, in order to ensure pearlescence of the preparation. In O/W emulsions, these fatty acid esters form ordered lamellar structures with lyotropic, liquid-crystalline properties. This leads to an optical property of the emulsions containing them which is referred to as pearlescence.

Preparations with pearlescence optics without the addition of these fatty acid esters are not accessible or accessible only with difficulty.

Cosmetic preparations and emulsions with pearlescence optics based on the emulsifier “stearic acid/palmitic acid” have been known for a long time. It was hitherto not possible to formulate pearlescent emulsions which have the neutralizing agent NaOH and fractions of fatty acids below 12% by weight.

In view of this, the pearlescent emulsions known from the prior art and available commercially exhibit very poor skin compatibility.

An optical pearlescent effect is achieved in cosmetic preparations according to the prior art exclusively by neutralization with triethanolamine or potassium hydroxide solution. With sodium hydroxide solution, no systems with mother of pearl-like optics could hitherto be prepared, as detailed, for example, in Modern Cosmeticology, Volume one, 1996, Ralph G. Harry, F.R.I.C, 1962, pp. 115-119. It becomes likewise clear therein that lipids and waxes inhibit the pearlescence in emulsions. Pearlescent emulsions are described which comprise very small, up to a maximum of 3% by weight, fractions of lipids and/or lipophilic consistency-imparting agents.

In addition, it is known that the sole use of sodium hydroxide solution in these systems does not ensure adequate storage stability of the cosmetic preparation, as explained, for example, in The American Perfumer, April 1945, “Manufacturing Vanishing Cream”, J. S. Shukla. It is also disclosed therein that pearlescent emulsions can be achieved exclusively by means of high use concentrations of fatty acids. Thus, for example, 16-25% by weight of fatty acids, where 13.3% by weight should be saponified, are used. However, it is also known, for example from Kosmetologie, 3rd edition, 1976, Dr. J. Jellinek (pp. 235-239), that precisely this high soap content of 13.3% by weight leads to poor skin compatibilities.

An object of the present invention is to provide a cosmetic preparation which has an optically pleasing effect, in particular a pearlescent effect, adequate storage stability and, in particular, good skin compatibility.

Moreover, the object of the present invention is to provide a cosmetic preparation which enriches the prior art.

The bundle of objects is achieved by a cosmetic preparation according to claim 1 or 2. The invention further comprises the use of the preparations according to the invention. Preferred embodiments of the preparations according to the invention are presented in the dependent claims.

Surprisingly, it has been found that cosmetic or dermatological preparations comprising
(I) up to 12% by weight, based on the total weight of the preparations, of one or more C₁₂-C₄₀ fatty acids,
(II) 0 to a maximum of 3% by weight, based on the total weight of the preparations, of one or more C₁₂-C₄₀ fatty alcohols
(III) 0.01 to 10% by weight, based on the total weight of the preparations, of amphiphilic polymers and/or associative polymers and/or siloxane elastomers and
(IV) sodium hydroxide solution
   or preparations comprising
(I) a maximum of 10% by weight, based on the total weight of the preparations, of one or more C₁₂-C₄₀ fatty acids,
(II) 0.1 to 10% by weight, based on the total weight of the preparations, of one or more C₁₂-C₄₀ fatty alcohols,
(III) 0.01 to 10% by weight, based on the total weight of the preparations, of amphiphilic polymers and/or associative polymers and/or siloxane elastomers,
(IV) sodium hydroxide solution and
(V) additionally C₁₂-C₄₀ polyethoxylated (POE) fatty acid esters with a POE chain length of 10-100
   enable all of the objects presented to be achieved.

It is characteristic that such pearlescent preparations in the sense of the present invention are advantageously free from mono- and/or di-fatty acid esters of glycerol and/or glycol. These customarily used emulsifiers and/or pearlescence-imparting agents are advantageously not used in order to ensure the pearlescent effect according to the invention in the above formulations. Particular preference is given to preparations according to the invention which contain no glyceryl stearate, glyceryl distearate, glyceryl isostearate, glyceryl diisostearate, glyceryl oleate, glyceryl palmitate, glyceryl myristate, glyceryl lanolate and/or glyceryl laurate.

The essential advantage of the preparations according to the invention lies in the possibility, shown for the first time, that sodium hydroxide solution may be present as exclusive neutralizing agent without accepting losses with regard to long-term stability, cosmetic pearlescence optics and, in particular, skin compatibility.

Suitable C₁₂-C₄₀ fatty acids (I) are completely neutralized, partially neutralized or unneutralized, branched and/or unbranched, saturated and/or unsaturated fatty acids with a chain length of from 12 to 40 carbon atoms.

The fatty acid(s) are preferably chosen from the group of acids which are completely or partially neutralized with customary alkalis (such as, for example, sodium and/or potassium hydroxide, sodium and/or potassium carbonate). For example, stearic acid and stearates, isostearic acid and isostearates, palmitic acid and palmitates, and myristic acid and myristates are particularly advantageous. Through the preparations according to the invention, it is possible for the first time to dispense with the use of mono- and/or triethanolamine as neutralizing agent. This achieves improved skin compatibility but good long-term stability and excellent pearlescence nevertheless.

Preference is given to using C16/C18 fatty acids and mixtures, in particular in a eutectic mixture, very particularly preferably stearic acid and palmitic acid.

In the fatty acids used, the saponified fraction is preferably a maximum of 9%.

The C₁₂-C₄₀ fatty acids (I) are used in an amount of up to 12% by weight, preferably from 0.1-10% by weight, based on the total preparation.

As C₁₂-C₄₀ fatty alcohols (II) are chosen alcohols from the group of saturated and/or unsaturated, branched and/or unbranched fatty alcohols with a chain length of from 12 to 40 carbon atoms. Preferably, C14-C20 fatty alcohols are chosen.

The fatty alcohols are preferably chosen according to the invention from the following group: myristyl alcohol behenyl alcohol (C₂₂H₄₅0H), cetearyl alcohol [a mixture of cetyl alcohol (C₁₆H₃₃0H) and stearyl alcohol (C₁₈H₃₇0H)], lanolin alcohols (wool wax alcohols, which represent the unsaponifiable alcohol fraction of wool wax which is obtained following saponification of wool wax). Particular preference is given to cetyl alcohol and cetylstearyl alcohol.

The C₁₂-C₄₀ fatty alcohols (II) are used in an amount of up to 10% by weight, preferably from 0.1-5% by weight, or up to 3% by weight, based on the total preparation.

As C₁₂-C₄₀ POE fatty acid esters (V) are chosen polyethoxylated fatty acid esters with a chain length of from 12 to 40 carbon atoms and with a degree of ethoxylation of from 10 to 100. From the group of polyethoxylated fatty acid esters, the following are preferably chosen: PEG-9 stearate, PEG-8 distearate, PEG-20 stearate, PEG-8 stearate, PEG-8 oleate, PEG-25 glyceryl trioleate, PEG-40 sorbitan lanolate, PEG-15 glyceryl ricinoleate, PEG-20 glyceryl stearate, PEG-20 glyceryl isostearate, PEG-20 glyceryl oleate, PEG-20 stearate, PEG-20 methylglucose sesquistearate, PEG-30 glyceryl isostearate, PEG-20 glyceryl laurate, PEG-30 stearate, PEG-30 glyceryl stearate, PEG-40 stearate, PEG-30 glyceryl laurate, PEG-50 stearate, PEG-100 stearate, PEG-150 laurate. Polyethoxylated stearic esters, for example, are particularly advantageous.

Particular preference is given to PEG-40 stearates.

The C₁₂-C₄₀ POE fatty acid esters (V) are used in an amount of up to 10% by weight, preferably from 0.1-5% by weight, based on the total preparation.

In one tried and tested preparation, the constituents I, II and V are in the ratio (I:II:V) 5:1:1 to 1:1:5. Ratios of I:II:V in the range 3:1:1 to 3:1:3 or in the range 3:1:1 to 1:1:3 are particularly preferred.

This targeted blending generates synergistic effects with regard to the positive properties of the cosmetic preparation.

As amphiphilic polymers and/or associative polymers (III) are chosen polymers which carry at least one fatty acid or fatty alcohol group, as the hydrophobic group, and a hydrophilic group. The polymers are water-soluble or can be dispersed in water as microgels. These polymers are referred to as swellable. The polymers can be composed of any chemical nature, e.g. free-radical polymers, vinyl or acrylic polymers, polycondensates and/or mixtures thereof. The polymers may have an ionic or nonionic structure, preference being given to anionic and nonionic polymers.

Preferably, as amphiphilic and/or associative polymers may be chosen:

Cellulose ethers containing hydrophobic substituents, such as alkyl groups with a carbon number greater than or equal to 8, such as, for example, hydroxyethylcellulose Natrosol Plus Grade 330 from Aqualon.

Quaternized cationic cellulose with at least one fatty acid group, such as alkyl, arylalkyl or alkylaryl groups groups or mixtures thereof, preferably with a carbon number of C₈-C₂₂. Quaternized alkylhydroxyethylcelluloses, as available, for example, under the name Quatrisoft LM 200, Quatrisoft LM-X 529-18-A, Quatrisoft LM-X 529-18-B (C₁₋₂ alkyl) and/or Quatrisoft LM-X 529-8 (C18 alkyl) from Amerchol. Preference is likewise given to quaternized alkylhydroxyethylcelluloses which are available under the names Crodacel QM, Crodacel QL (C₁₂ alkyl) and/or Crodacel QS (C₁₈ alkyl) from Croda.

Galactomannans containing hydrophobic substituents, in particular the derivatives as are disclosed in EP-A-281360.

Pullulans modified by hydrophobic groups, in particular cholesterol groups.

Gelatin modified by hydrophobic groups, in particular C6-C18 alkyl groups; Mucopolysaccharides obtained from glycosaminoglycans and hyaluronic acid.

In addition, those associative and/or amphiphilic polymers as are disclosed in EP-B1-1046387, which are hereby explicitly included in the disclosure content of the present invention, can be preferably used.

Particularly suitable associative polymers have proven to be those chosen from the group:
of polyethylene glycol ethers of the general formula R—O—(—CH₂—CH₂—O—)ₙ—R′, where R and R′, independently of one another, are branched or unbranched alkyl, aryl or alkenyl radicals and n is a number greater than 100,
of etherified fatty acid ethoxylates of the general formula R—COO—(—CH₂—CH₂—O—)ₙ—R′, where R and R′, independently of one another, are branched or unbranched alkyl, aryl or alkenyl radicals and n is a number greater than 100,
of esterified fatty acid ethoxylates of the general formula R—COO—(—CH₂—CH₂—O—)ₙ—C(O)—R′, where R and R′, independently of one another are branched or unbranched alkyl, aryl or alkenyl radicals and n is a number greater than 100, of polypropylene glycol ethers of the general formula R—O—(—CH₂—CH(CH₃)—O—)ₙ—R′, where R and R′, independently of one another, are branched or unbranched alkyl, aryl or alkenyl radicals and n is a number greater than 100, of esterified fatty acid propoxylates of the general formula R—COO—(—CH₂—CH(CH₃)—O—)ₙ—C(O)—R′, where R and R′, independently of one another, are branched or unbranched alkyl, aryl or alkenyl radicals and n is a number greater than 100, of polypropylene glycol ethers of the general formula R—O—Xₙ—Yₘ—R′, where R and R′, independently of one another, are branched or unbranched alkyl, aryl or alkenyl radicals, where X and Y are not identical and are in each case either an oxyethylene group or an oxypropylene group and n and m, independently of one another, are numbers, the sum of which is greater than 100
of etherified fatty acid propoxylates of the general formula R—COO—Xₙ—Yₘ—R′, where R and R′, independently of one another, are branched or unbranched alkyl, aryl or alkenyl radicals, where X and Y are not identical and are in each case either an oxyethylene group or an oxypropylene group and n and m, independently of one another, are numbers, the sum of which is greater than 100
tetramethoxymethylglycoluril

Advantageous polymers according to the invention are characterized in particular by the following structural formula 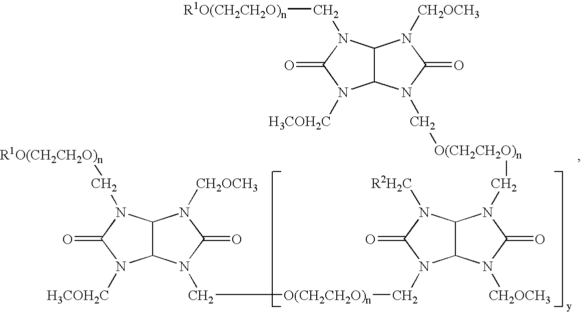
in which
R¹ is a branched or unbranched, saturated or unsaturated alkyl radical having 4 to 40 carbon atoms,
R²=—OCH₃ or —O(CH₂CH₂O)ₓR¹,
x is an integer from 1 to 100,
n is an integer from 100 to 250 and
y is on average 2 or 3.

Particularly advantageous polymers according to the invention are those for which n is an integer from 150 to 200. It is particularly advantageous in the sense of the present invention if, moreover, R¹ is a branched or unbranched, saturated or unsaturated alkyl radical having 8 to 12 carbon atoms.

It is also advantageous in the sense of the present invention if the average molar mass of the polymers is between 30,000 and 50,000.

According to the invention, PEG-180/octoxynol-40/tetramethoxymethylglycoluril copolymers where R²=—O(CH₂CH₂O)₄₀C₈H₁₇ and n=180 are very particularly advantageous.

Also particularly advantageous according to the invention are PEG-180/laureth-50/tetra-methoxymethylglycoluril copolymers where R²=—O(CH₂CH₂O)₅₀C₁₂H₂₅ and n=180.

According to the invention, polyether-1 is also particularly advantageous.

PEG-150 distearate and PEG-150 dioleate are particularly advantageous. PEG-300 pentaerythrityl tetraisostearate, PEG-120 methylglucose dioleate, PEG-160 sorbitan triisostearate, PEG-450 sorbitol hexaisostearate and PEG-230 glyceryl triisostearate are also to be used advantageously as associative polymers.

It is accordingly also advantageous to choose hydrophobically substituted polysaccharide derivatives as associative thickeners, for example hydrophobically substituted cellulose ethers, hydrophobically substituted starches, alginates, glucans, chitins, dextrans, caseinates, pectins, proteins and gums, and also polyurethanes, polyacrylamides, polyvinyl alcohols, polyacrylates and the like.

The hydrophobically substituted polysaccharide derivatives described in US patent specification 5,426,182, which are hereby explicitly included in the disclosure content of the present invention, are particularly advantageous.

It may also in some instances be advantageous if the associative polymer or polymers used according to the invention has or have physiological effectiveness within the meaning of a cosmetic or pharmaceutical effect. Thus, for example, the biosurfactant esters disclosed in DE-A1-4344661 can advantageously be used in the sense of the present invention.

As preferred are to be mentioned nonionic polymers such as Pure Thix TX grades, crosspolymers, such as acrylates/vinyl isodecanoate crosspolymer (Stabylen 30 from 3-V-Sigma) and acrylates/C10-C30 alkyl acrylate crosspolymer (Pemulen TR 1, Pemulen TR 2, Ultrez 21, Carbopol ETD 2020, Carbopol ETD 2001 from Noveon), and hydrophobically modified polyacrylates (HASE grades), such as acrylates/steareth-20 methacrylate copolymer (Acrysol and/or Aculyn 22 from Rohm & Haas), acrylates/steareth-20 itaconate copolymer (Structure 2000 from National Starch).

It is advantageous in the sense of the present invention to choose the total amount of the associative and/or amphiphilic polymers from the range from 0.01 to 5% by weight, advantageously from 0.1 to 1% by weight, in each case based on the total weight of the formulation.

The preferred amphiphilic polymers are listed in Table 1 below, which also lists their trade name besides the structural formula and the INCI name.

TABLE 1

| INCl | Structural formula | Trade name |
|---|---|---|
| Acrylates/ Steareth-20 Methacrylate Copolymer | 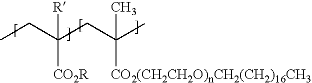 | Acrysol 22 Polymer (Rohm + Haas) |
| Acrylates/ Steareth-20 Itaconate Copolymer | 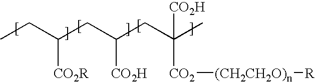 | Structure 2000 (National Starch) |
| Steareth-10 Ally Ether/ Acrylate Copolymer | 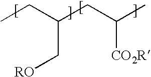 | Salcare SC 80 (30% strength) |
| Acrylates/ Steareth-50 Acrylate Copolymer | 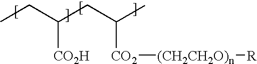 | Antil 208 (Goldschmidt) (Mixture with Laureth-3, Propylene (Glycol) |
| Acrylates/ Palmeth-25 Acrylate Copolymer | | Synthalen W 2000 |
| Acrylates/ C10-30 Alkyl Acrylates Crosspolymer | 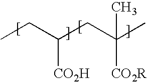 | Pemulen TR-1 Pemulen TR-2, (Noveon) |
| Acrylates/ Vinyl Isodecanote Crosspolymer | | Stabylen 30 |
| Cetylhydroxyethyl- cellulose | 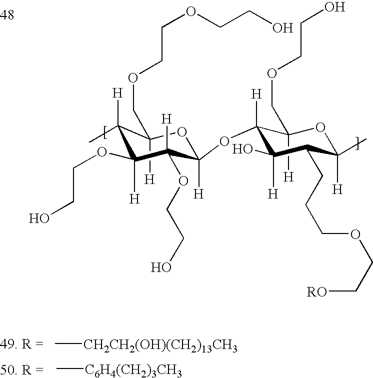 | Natrosol Plus 330 CS (Hercules) |
| other alkyl-modified cellulose derivatives | | |
| “Polyquaternium-24” | | Quatrisoft Polymer LM-200 (Amerchol) |
| PEG-120 Methylglucose Dioleate | 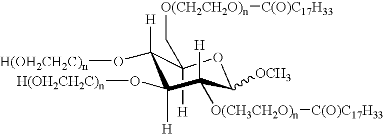 | Glucamate DOE-120 (Nordmann & Rassmann) |
| PEG-60 Sorbitan Tetraoleate | 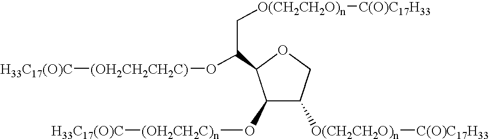 | Nikkol GO-460 (Nikko chemicals.) |
| PEG-150 Pentaerythrityl Tetrastearate | 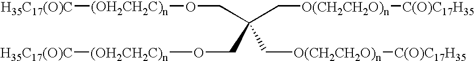 | Crothix, Code ES 2054 (Croda) |
| PEG-55 Propylene Glycol Oleate | 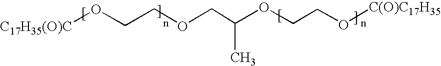 | Antil 141 Liquid Goldschmidt (40% Polymer, 40% Propylene Glycol, 20% water) |
| PEG-180/ Laureth- 50/TMMG Copolymer | 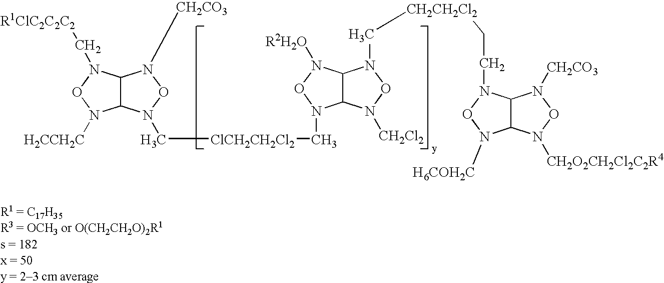 | Pure Thix TX 1450 (Süd-Chemie) |
| PEG-150 Distearate | 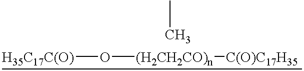 | Rewopal PEG 6000 DS |

The use of solid elastomeric polyorganosiloxanes or organopolysiloxanes, referred to below as siloxane elastomers, in cosmetic preparations is known per se and has achieved importance in recent years. Besides being used in cosmetics, these substances have been used in foods and animal feeds, pharmaceuticals, impregnating agents, lubricants and so on. Siloxane elastomers are partially or completely crosslinked and in most cases have a three-dimensional structure. They are obtainable by a reaction of vinyl-terminated polymethylsiloxane and methylhydrodimenthylsiloxane or else by reaction of hydroxy-terminated dimethylpolysiloxane and trimethylsiloxy-terminated methylpolysiloxane:

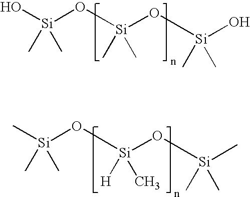

These siloxane elastomers are used, for example, for adjusting the rheological properties of a preparation. Siloxane elastomers of this type are described, for example, in the European patent specification 295886, and the US patent specification 5,266,321, which disclose the use of these substances in face-cleansing compositions and oil-containing makeup products. These specifications also describe the nature of siloxane elastomers in more detail. The siloxane elastomers are used in cosmetic preparations in particular due to their pleasant sensory properties, the resulting products are described as velvety, powdery and/or matting. In addition, they have stabilizing effects on formulations with a high content of oil and low water contents of at most 5% by weight. When formulating the abovementioned products, the problem of the siloxane elastomers being incompatible with other frequently used components often arises, which leads to unsatisfactory long-term stability of the products.

These disadvantages known from the prior art have been overcome according to the invention.

In this connection it is preferred when siloxane elastomers chosen from the groups of siloxane elastomers are used (a) which contain units R₂SiO and RSiO_{1.5} and/or R3SiO_{0.5} and/or SiO₂, where the individual radicals R are, in each case, independently of one another, hydrogen, alkyl, such as, for example, methyl, ethyl, propyl or aryl, such as, for example, phenyl or tolyl, alkenyl, such as, for example, vinyl, and the weight ratio of the units R₂SiO to RSiO_{1.5} is chosen in the range from 1:1 to 30:1; (b) which are insoluble and swellable in silicone oil and which are obtainable by the addition reaction of an organopolysiloxane (1) which contains silicon-bonded hydrogen with an organopolysiloxane (2) which contains unsaturated aliphatic groups, where the quantitative ratios used are chosen such that the amount of the hydrogen of the organopolysiloxane (1) or of the unsaturated aliphatic groups of the organopolysiloxane (2) is in the range from 1 to 20 mol % if the organopolysiloxane is not cyclic, and is in the range from 1 to 50 mol % if the organopolysiloxane is cyclic. It is particularly preferred when the organopolysiloxane elastomer is used in combination with oils of hydrocarbons of animal and/or vegetable origin, synthetic oils, synthetic esters, synthetic ethers or mixtures thereof. It is very particularly preferred when the organopolysiloxane elastomer is used in combination with unbranched silicone oils which are liquid or pasty at room temperature, or cyclic silicone oils or mixtures thereof. It is very exceptionally preferred when the organopolysiloxane elastomer is used in the form of a gel of organopolysiloxane elastomer and a lipid phase, where the content of the organopolysiloxane elastomer in the gel is 3 to 80% by weight, very exceptionally preferably 0.3 to 60% by weight.

It is advantageous to use siloxane elastomers from Dow Corning, as are described in the US American patent specification 5,654,362 and are available under the trade name 9040 Silicone Elastomer Blend, in the formulations according to the invention. Likewise advantageous are siloxane elastomers from Grant Chemical with the INCI name Polysilicone-11, such as the grades Gransil GCM or Gransil PM, as are described in the US American patent specifications 5,266,321, 4,980,167 and 4,742,142. Particularly advantageous siloxane elastomers are also those which are present in the form of spherical powders with an average particle size of from 2 to 5 μm and a particle size distribution of from 1 to 15 μm and are described in the Japanese patent specifications 4-66446 and 4-17162, and in the Japanese laid-open specification 2-243612 or are described with an average particle size of less than 50 μm in the specifications EP 0295 886 and U.S. Pat. No. 4,761,454. Commercially available products are, for example, Torayfil E-505C, Torayfil E-506 C from Toray-Dow Corning Silicone Co.

A particularly advantageous siloxane elastomer in the form of spherical powder is dimethicone/vinyldimethicone crosspolymer with a particle size distribution of from 1 to 15 μm, available under the trade name Dow Corning 9509 Powder from Dow Corning. Also advantageous are the gels that contain the siloxane elastomers which are available under the trade names KSG-15, -16, -17, -18, -20 from Shin-Etsu or Gransil 5CYCgel, Gransil SR DMF 10gel, Gransil SR DC 556gel from Grant Chemical, SF 839, SF1204, JK113 from General Electric, and lauryl dimeticone/vinyldimethicone crosspolymers, as are available under the names KSG41, -42, -43, -44 from Shin-Etsu. A further advantageous siloxane elastomer is the cyclomethicone+vinyldimethicone/methicone crosspolymer or the chemically related elastomer Crosslinked Stearyl Methyl Dimethyl Siloxane Elastomer as are supplied, for example, by Grant Chemical under the name SR-CYC.

The siloxane elastomers used are also emulsions and/or suspensions which comprise siloxane elastomers. By way of example, preference is given to the emulsions and/or suspensions in Table 2 below.

TABLE 2

| Trade name/ | | |
|---|---|---|
| BDF | INCI | Elastomer content |
| Dow Corning | Cyclopentasiloxane (and) | 70.6% DC 9040; |
| 9040 Emulsion/ | Dimethicone Crosspolymer | 10.8% DC345; 5.4% |
| Dow Corning | (and) Cyclohexasiloxane (and) | DC 200; 1.9% Brij35; |
| 9040 | Cyclomethicone (and) | 0.8% Brij30; |
| Concentrated | Dimethicone (and) Laureth-4 | 0.55% Liquapar Optima; |
| Emulsion | (and) Laureth-23 (and) | 9.95% Water |
| | Parabens*) | |
| Dow Corning | Dimethicone/Vinyl | 63% spherical siloxane |
| 9509 Silicone | Dimethicone Crosspolymer + | elastomers in water |
| Elastomer | C12-14 Pareth-12 | |
| Suspension/ | | |
| Dow Corning | | |
| 9509 | | |

The siloxane elastomers (III) are used in an amount of up to 10% by weight, preferably of 0.5-10% by weight of the pure elastomer, based on the total preparation.

In addition, low molecular weight surfactants (VI), in particular ceteareth-3, ceteareth-6, steareth-2, steareth-10, ceteth-10, isosteareth-10, laureth-2, laureth-3, laureth-4, myreth-4, laneth-5, ceteth-2, ceteth-3, oleth-2, oleth-3 and/or oleth-5 are preferably added to the preparations according to the invention. Laureth-4 is very particularly preferred.

Particularly advantageous preparations are the following formulations, which comprise
(I) up to 10% by weight of stearic acid/palmitic acid,
(II) 0.1-10% by weight of cetyl alcohol, behenyl alcohol, stearyl alcohol and/or cetearyl alcohol
(III) 0.01-10% by weight of dimethicone/vinyl dimethicone crosspolymer, polysilicone-11, acrylate/alkyl acrylate crosspolymer, acrylate/vinyl isodecanoate crosspolymer, acrylate/steareth-20 methacrylate copolymer, acrylate/steareth-20 itaconate copolymer, acrylate/steareth-50 acrylate copolymer, acrylate/palmeth-25 acrylate copolymer, steareth-10 allyl ether/acrylate copolymer, PEG-120 methylglucose dioleate, PEG-60 sorbitan tetraoleate, PEG-150 pentaerythrityl tetrastearate, PEG-55 propylene glycol oleate, PEG-150 distearate and/or PEG-180/laureth-50/TMMG copolymer
(IV) 0.15-1% by weight of sodium hydroxide solution
(V) up to 10% by weight of PEG-20 stearate, PEG-40 stearate and/or PEG-100 stearate and
(VI) optionally up to 10% by weight of steareth-2, laureth-4 and/or ceteth-3.

Preparations of the following formulations have likewise proven to be advantageous
(I) up to 12% by weight of stearic acid/palmitic acid,
(II) 0-3% by weight of cetyl alcohol, behenyl alcohol, stearyl alcohol and/or cetearyl alcohol
(III) 0.01-10% by weight of dimethicone/vinyl dimethicone crosspolymer, polysilicone-11, acrylate/alkyl acrylate crosspolymer, acrylate/vinyl isodecanoate crosspolymer, acrylate/steareth-20 methacrylate copolymer, acrylate/steareth-20 itaconate copolymer, acrylate/steareth-50 acrylate copolymer, acrylate/palmeth-25 acrylate copolymer, steareth-10 ally ether/acrylate copolymer, PEG-120 methylglucose dioleate, PEG-60 sorbitan tetraoleate, PEG-150 pentaerythrityl tetrastearte, PEG-55 propylene glycol oleate, PEG-150 distearate and/or PEG-180/laureth-50/TMMG copolymer,
(IV) 0.25-1% by weight of sodium hydroxide solution

It may be advantageous, although it is not necessary, for the formulations according to the present invention to comprise further emulsifiers. Preference is given to using those emulsifiers which are suitable for the preparation of W/O emulsions, it being possible for these to be present either individually or else in any combinations with one another.

The further emulsifier(s) is/are advantageously chosen from the group which comprises the following compounds:

polyglyceryl-2 dipolyhydroxystearate, PEG-30 dipolyhydroxystearate, cetyldimethicone copolyol, glycol distearate, glycol dilaurate, diethylene glycoldilaurate, sorbitan trioleate, glycol oleate, glyceryl dilaurate, sorbitan tristearate, propylene glycol stearate, propylene glycol laurate, propylene glycol distearate, sucrose distearate, PEG-3 castor oil, pentaerythrityl monostearate, pentaerythrityl sesquioleate, glyceryl oleate, pentaerythrityl monooleate, sorbitan sesquioleate, isostearyl diglyceryl succinate, glyceryl caprate, palm glycerides, cholesterol, lanolin, glyceryl oleate (with 40% monoester), polyglyceryl-2 sesquiisostearate, polyglyceryl-2 sesquioleate, PEG-20 sorbitan beeswax, sorbitan oleate, sorbitan isostearate, trioleyl phosphate, glyceryl stearate and ceteareth-20 (Teginacid from Th. Goldschmidt), sorbitan stearate, PEG-7 hydrogenated castor oil, PEG-5 soy sterol, PEG-6 sorbitan beeswax, methylglucose sesquistearates, PEG-10 hydrogenated castor oil, sorbitan palmitate, PEG-22/dodecyl glycol copolymer, polyglyceryl-2 PEG-4 stearate, sorbitan laurate, PEG-4 laurate, polysorbate 61, polysorbate 81, polysorbate 65, polysorbate 80, triceteareth-4 phosphate, and sodium C₁₄₋₁₇ alkyl sec sulphonate (Hostacerin CG from Hoechst), polysorbate 85, trilaureth-4 phosphate, PEG-35 castor oil, sucrose stearate, trioleth-8 phosphate, C₁₂₋₁₅ pareth-12, PEG-40 hydrogenated castor oil, PEG-16 soy sterol, polysorbate 80, polysorbate 20, polyglyceryl-3 methylglucose distearate, PEG-40 castor oil, sodium cetearyl sulphate, lecithin, laureth-4 phosphate, propylene glycol stearate SE, PEG-25 hydrogenated castor oil, PEG-54 hydrogenated castor oil, PEG-6 caprylic/capric glycerides, glyceryl oleate and propylene glycol, polysorbate 60, polyglyceryl-3 oleate, PEG-40 sorbitan peroleate, laureth-4, isostearyl glyceryl ether, cetearyl alcohol and sodium cetearyl sulphate, PEG-22 dodecyl glycol copolymer, polyglyceryl-2 PEG-4 stearate, pentaerythrityl isostearate, polyglyceryl-3 diisostearate, sorbitan oleate and hydrogenated castor oil and Cera alba and stearic acid, sodium dihydroxycetyl phosphate and isopropyl hydroxycetyl ether, methylglucose sesquistearate, methylglucose dioleate, sorbitan oleate and PEG-2 hydrogenated castor oil and ozokerite and hydrogenated castor oil, PEG-2 hydrogenated castor oil, PEG-45/dodecyl glycol copolymer, methoxy PEG-22/dodecyl glycol copolymer, hydrogenated cocoglycerides, polyglyceryl-4 isostearate, PEG-40 sorbitan peroleate, PEG-40 sorbitan perisostearate, PEG-8 beeswax, laurylmethicone copolyol, polyglyceryl-2 laurate, stearamidopropyl PG dimonium chloride phosphate, PEG-7 hydrogenated castor oil, triethyl citrate, glyceryl stearate citrate, cetyl phosphate, polyglycerol methyl-glucose distearate, poloxamer 101, potassium cetyl phosphate, polyglyceryl-3 diisostearates and/or AbilCare 85 from Dow Corning.

Preferably, in the sense of the present invention, the further emulsifier(s) is/are chosen from the group of hydrophilic emulsifiers. According to the invention, particular preference is given to mono-, di- and tri-fatty acid esters of sorbitol.

The total amount of the further emulsifiers is, according to the invention, advantageously chosen to be less than 5% by weight, based on the total weight of the formulation.

The list of the given further emulsifiers which can be used in the sense of the present invention is not of course intended to be limiting.

Particularly advantageous pearlescent preparations in the sense of the present invention are free from mono- and/or di-fatty acid esters of glycerol and/or glycol. These customarily used emulsifiers are advantageously not used in accordance with the invention in order to ensure the pearlescent effect according to the invention.

Particular preference is given to preparations according to the invention which comprise no glyceryl stearate, glyceryl isostearate, glyceryl diisostearate, glyceryl oleate, glyceryl palmitate, glyceryl myristate, glyceryl lanolate and/or glyceryl laurate.

In addition, solubilizers may be present in the preparations, such as, for example, PEG-40 hydrogenated castor oil. The advantage of the solubilizers is that they promote the pearlescence structures at elevated temperatures and consequently generate an additional aesthetic benefit. As suitable solubilizers, polysorbate 80, polysorbate 60, PEG-40 castor oil, PEG-40 hydrogenated castor oil, PEG-60 hydrogenated castor oil, polyglyceryl-3 laurate, PEG-20 glyceryl laurate, methyl gluceth-20, nonoxynol-10, PPG-1 PEG-9 lauryl glycol ether, ceteth-16, PEG-16 soy sterol, PEG-10 soy sterol, C12-15 pareth-12, nonoxynol-14, octoxynol-16, PEG-20 glyceryl stearate, sorbeth-30, PPG-26 buteth-26+PEG-40 hydrogenated castor oil, tri C12-13 alkyl citrates, polyglyceryl-2 isostearates, polyglyceryl-2 diisostearates, PPG-15 stearyl ether, PEG-10 olive glycerides, PPG-3 methyl ether, PEG-2 diethyl hexanoate, C20-40 pareth-40, PEG-60 almond glycerides and/or PEG-6 caprylic/capric glycerides may be chosen.

In addition, nonpolar lipids, mineral oils, silicone oils and/or waxes may be present in the cosmetic formulations in an amount up to 30% by weight, based on the total mass of the preparation.

The nonpolar lipids or waxes are preferably chosen from the group of nonpolar hydrocarbons, hydrogenated polyisobutene, cyclomethicones, dimethicones, methyl palmitate and/or dimethiconol stearate.

In the sense of the present disclosure, the expression “lipids” is sometimes used as the generic term for fats, oils, waxes and the like, as is entirely familiar to a person of skill in the art. The terms “oil phase” and “lipid phase” are also used synonymously.

Oils and fats differ from one another, inter alia, in their polarity, which is difficult to define. It has already been proposed to adopt the interfacial tension towards water as a measure of the polarity index of an oil or of an oil phase. This means that the lower the interfacial tension between this oil phase and water, the greater the polarity of the oil phase in question. According to the invention, the interfacial tension is regarded as one possible measure of the polarity of a given oil component.

The interfacial tension is the force which acts on an imaginary line one metre in length in the interface between two phases. The physical unit for this interfacial tension is conventionally calculated from the force/length relationship and is usually expressed in mN/m (millinewtons divided by metres). It has a positive sign if it endeavours to reduce the interface. In the converse case, it has a negative sign. In the sense of the present invention, polar lipids are regarded as being lipids whose interfacial tension towards water is less than 20 mN/m, and nonpolar lipids are regarded as being those whose interfacial tension towards water is more than 30 mN/m. Lipids with an interfacial tension towards water between 20 and 30 mN/m are generally referred to as mid-polar.

Particularly advantageous mid-polar lipids in the sense of the present invention are the substances listed below:

| | | | Polarity |
|---|---|---|---|
| Manufacturer | Trade name | INCI name | mN/m |
| Stearinerie | DUB VCI 10 | Isodecyl Neopentanoate | 29.9 |
| Dubois Fils | | | |
| ALZO (ROVI) | Dermol IHD | Isohexyl Decanoate | 29.7 |
| ALZO (ROVI) | Dermol 108 | Isodecyl Octanoate | 29.6 |
| | Dihexyl Ether | Dihexyl Ether | 29.2 |
| ALZO (ROVI) | Dermol 109 | Isodecyl 3,5,5 Trimethyl | 29.1 |
| | | Hexanoate | |
| Henkel Cognis | Cetiol SN | Cetearyl Isononanoate | 28.6 |
| Unichema | Isopropyl | Isopropyl Palmitate | 28.8 |
| | palmitate | | |
| Dow Corning | DC Fluid 345 | Cyclomethicone | 28.5 |
| Dow Corning | Dow Corning | Cyclopolydimethylsiloxane | 28.5 |
| | Fluid 244 | | |
| Nikko | Jojoba oil Gold | | 26.2 |
| Chemicals | | | |
| Superior Jojoba | | | |
| Oil Gold | | | |
| Wacker | Wacker AK 100 | Dimethicone | 26.9 |
| ALZO (ROVI) | Dermol 98 | 2-Ethylhexanoic Acid 3,5,5 | 26.2 |
| | | Trimethyl Ester | |
| Dow Corning | Dow Corning | Open | 25.3 |
| | Fluid 246 | | |
| Henkel Cognis | Eutanol G | Octyldodecanol | 24.8 |
| Condea | Isofol 16 | Hexyl Decanol | 24.3 |
| Chemie | | | |
| ALZO (ROVI) | Dermol 139 | Isotridecyl 3,5,5 | 24.5 |
| | | Trimethylhexanonanoate | |
| Henkel Cognis | Cetiol PGL | Hexyldecanol (+) | 24.3 |
| | | Hexyl Decyl Laurate | |
| | Cegesoft C24 | Octyl Palmitate | 23.1 |
| Gattefossé | M.O.D. | Octyldodeceyl Myristate | 22.1 |
| | Macadamia Nut | | 22.1 |
| | Oil | | |
| Bayer AG, | Silicone oil VP | Phenyl Trimethicone | 22.7 |
| Dow Corning | 1120 | | |
| CONDEA | Isocarb 12 | Butyl Octanoic Acid | 22.1 |
| Chemie | | | |
| Henkel Cognis | Isopropyl stearate | Isopropyl Stearate | 21.9 |
| WITCO, | Finsolv TN | C12-15 Alkyl Benzoate | 21.8 |
| Goldschmidt | | | |
| Dr. Straetmans | Dermofeel BGC | Butylene Glycol | 21.5 |
| | | Caprylate/Caprate | |
| Unichema | Miglyol 812 | Caprylic/Capric | 21.3 |
| Huels | | Triglyceride | |
| Trivent (via S. | Trivent OCG | Tricaprylin | 20.2 |
| Black) | | | |
| ALZO (ROVI) | Dermol 866 | PEG Diethylhexanoate/ | 20.1 |
| | | Diisononanoate/Ethylhexyl | |
| | | Isononanoate | |

Particular preference is given to nonpolar lipids. Nonpolar oils are, for example, those which are chosen from the group of branched and unbranched hydrocarbons and hydrocarbon waxes, in particular vaseline (petrolatum), paraffin oil, squalane and squalene, polyolefins and hydrogenated polyisobutenes. Among the polyolefins, poly-decenes are the preferred substances.

Particularly advantageous nonpolar lipids in the sense of the present invention are the substances listed below:

| | | | Polarity |
|---|---|---|---|
| Manufacturer | Trade name | INCI name | mN/m |
| Total SA | Ecolane 130 | Cycloparaffin | 49.1 |
| Neste PAO N.V. | Nexbase 2006 FG | Polydecene | 46.7 |
| (Supplier Hansen & Rosenthal) | | | |
| Chemische Fabrik Lehrte | Polysynlane | Hydrogenated | 44.7 |
| | | Polyisobutene | |
| Wacker | Wacker Silicone oil | Polydimethylsiloxane | 46.5 |
| | AK 50 | | |
| EC Erdölchemie (Supplier Bayer | Solvent ICH | Isohexadecane | 43.8 |
| AG) | | | |
| DEA Mineral oil (Supplier Hansen | Pionier 2076 | Mineral Oil | 43.7 |
| & Rosenthal) Tudapetrol | | | |
| DEA Mineral oil (Supplier Hansen | Pionier 6301 | Mineral Oil | 43.7 |
| & Rosenthal) Tudapetrol | | | |
| Wacker | Wacker Silicone oil | Polydimethylsiloxane | 42.4 |
| | AK 35 | | |
| EC Erdölchemie GmbH | Isoeicosane | Isoeicosane | 41.9 |
| Wacker | Wacker Silicone oil | Polydimethylsiloxane | 40.9 |
| | AK 20 | | |
| Condea Chemie | Isofol 1212 | | 40.3 |
| | Carbonate | | |
| Gattefossé | Softcutol O | Ethoxydiglycol Oleate | 40.5 |
| Creaderm | Lipodermanol OL | Decyl Olivate | 40.3 |
| Henkel | Cetiol S | Dioctylcyclohexane | 39.0 |
| DEA Mineral oil (Supplier Hansen | Pionier 2071 | Mineral Oil | 38.3 |
| & Rosenthal) Tudapetrol | | | |
| WITCO BV | Hydrobrite 1000 PO | Paraffinum Liquidum | 37.6 |
| Goldschmidt | Tegosoft HP | Isocetyl Palmitate | 36.2 |
| Condea Chemie | Isofol Ester 1693 | | 33.5 |
| Condea Chemie | Isofol Ester 1260 | | 33.0 |
| Dow Corning | Dow Corning Fluid | Cyclopentasiloxane | 32.3 |
| | 245 | | |
| Unichema | Prisorine 2036 | Octyl Isostearate | 31.6 |
| Henkel Cognis | Cetiol CC | Dicaprylyl Carbonate | 31.7 |
| ALZO (ROVI) | Dermol 99 | Trimethylhexyl | 31.1 |
| | | Isononanoate | |
| ALZO (ROVI) | Dermol 89 | 2-Ethylhexyl | 31.0 |
| | | Isononanoate | |
| Henkel Cognis | Cetiol OE | Dicaprylyl Ether | 30.9 |
| | Dihexyl carbonate | Dihexyl Carbonate | 30.9 |
| Albemarle S.A. | Silkflo 366 NF | Polydecene | 30.1 |
| Unichema | Estol 1540 EHC | Octyl Cocoate | 30.0 |

It is, however, also advantageous to use mixtures of high-polarity and low-polarity lipids and the like. For example, the oil phase can advantageously be chosen from the group of branched and unbranched hydrocarbons and hydrocarbon waxes, dialkyl ethers, the group of Guerbet alcohols, such as, for example, octyldodecanol, the group of saturated or unsaturated, branched or unbranched alcohols, and of fatty acid triglycerides, namely the triglycerol esters of saturated and/or unsaturated, branched and/or unbranched alkanecarboxylic acids with a chain length of from 8 to 24, in particular 12-18, carbon atoms. The fatty acid triglycerides can, for example, advantageously be chosen from the group of synthetic, semisynthetic and natural oils, e.g. olive oil, sunflower oil, soybean oil, peanut oil, rapeseed oil, almond oil, palm oil, coconut oil, palm kernel oil and the like, provided the conditions required in the main claims are observed.

Any blends of oil and wax components are also to be used advantageously in the sense of the present invention. It may also in some cases be advantageous to use waxes, for example cetyl palmitate, as the sole lipid component of the oil phase.

Fatty and/or wax components to be used advantageously according to the invention can be chosen from the group of vegetable waxes, animal waxes, mineral waxes and petrochemical waxes. Favourable according to the invention are, for example, candelilla wax, carnauba wax, Japan wax, espartograss wax, suberic wax, guaruma wax, rice germ oil wax, sugar cane wax, berry wax, ouricury wax, montan wax, jojoba wax, shea butter, beeswax, shellac wax, spermaceti, lanolin (wool wax), uropygial grease, ceresin, ozokerite (earth wax), paraffin waxes and microwaxes, provided the conditions required in the main claim are observed.

Further advantageous fatty and/or wax components are chemically modified waxes and synthetic waxes, such as, for example, those available under the trade names Syncrowax HRC (glyceryl tribehenate), and Syncrowax AW 1C(C₁₈₋₃₆-fatty acid) from CRODA GmbH, and montan ester waxes, sasol waxes, hydrogenated jojoba waxes, synthetic or modified beeswaxes (e.g. dimethicone copolyol beeswax and/or C₃₀₋₅₀-alkyl beeswax), polyalkylene waxes, polyethylene glycol waxes, but also chemically modified fats, such as, for example, hydrogenated plant oils (for example hydrogenated castor oil and/or hydrogenated coconut fatty glycerides), triglycerides, such as, for example, trihydroxystearin, fatty acids, fatty acid esters and glycol esters, such as, for example, C₂₀₋₄₀-alkyl stearate, C₂₀₋₄₀-alkylhydroxystearoyl stearate and/or glycol montanate. Further advantageous are also certain organosilicon compounds which have similar physical properties to the specified fatty and/or wax components, such as, for example, stearoxytrimethylsilane.

Particularly advantageous fatty and/or wax components are chemically modified waxes and synthetic waxes, such as, for example, those available under the trade names Ultrasil IWS (dimethiconol stearate) from Noveon, and Estol 1503 (methyl palmitate) from Uniqema.

The oil phase is advantageously chosen from the group consisting of 2-ethylhexyl isostearate, octyldodecanol, isotridecyl isononanoate, butylene glycol dicaprylate/dicaprate, 2-ethylhexyl cocoate, C₁₂₋₁₅-alkyl benzoate, caprylic/capric triglyceride, dicaprylyl ether.

Mixtures of octyldodecanol, caprylic/capric triglyceride, dicaprylyl ether, dicaprylyl carbonate, cocoglycerides, or mixtures of C₁₂₋₁₅-alkyl benzoate and 2-ethylhexyl isostearate, mixtures of C₁₂₋₁₅-alkyl benzoate and butylene glycol dicaprylate/dicaprate and mixtures of C₁₂₋₁₅-alkyl benzoate, 2-ethylhexyl isostearate and isotridecyl isononanoate are particularly advantageous.

Of the hydrocarbons, paraffin oil, cycloparaffin, squalane, squalene, hydrogenated polyisobutene and polydecene are to be used advantageously in the sense of the present invention, provided the conditions required in the main claims are observed.

It may likewise be advantageous to choose the oil phase of the preparations according to the invention in part or entirely from the group of cyclic and/or linear silicones, which are also referred to in the sense of the present disclosure as “silicone oils”. Such silicones or silicone oils may be present as monomers which are generally characterized by structural elements, as follows:

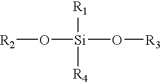

Silicone oils are high molecular weight synthetic polymeric compounds in which silicon atoms are joined in a chain-like and/or reticular manner via oxygen atoms and the remaining valences of the silicon are saturated by hydrocarbon radicals (in most cases methyl groups, less often ethyl, propyl, phenyl groups, etc.).

Linear silicones having a plurality of siloxyl units which are to be used advantageously according to the invention are generally characterized by structural elements, as follows: 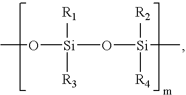
where the silicon atoms can be substituted by identical or different alkyl radicals and/or aryl radicals, which are shown here in general terms by the radicals R₁-R₄ (that is to say that the number of different radicals is not necessarily limited to 4). m can assume values from 2-200,000.

Systematically, the linear silicone oils are referred to as polyorganosiloxanes; the methyl-substituted polyorganosiloxanes, which represent the most important compounds of this group in terms of amount and are characterized by the following structural formula 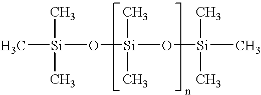
are also referred to as polydimethylsiloxane or dimethicone (INCI). Dimethicones are available in various chain lengths and with various molecular weights. Dimethicones of varying chain length and phenyltrimethicones are particularly advantageous linear silicone oils in the sense of the present invention.

Particularly advantageous polyorganosiloxanes in the sense of the present invention are also, for example, dimethylpolysiloxanes [poly(dimethylsiloxane)], which are available, for example, under the trade names ABIL 10 to 10 000 from Th. Goldschmidt. Also advantageous are phenylmethylpolysiloxanes (INCI: Phenyl Dimethicone, Phenyl Trimethicone), cyclic silicones (octamethylcyclotetrasiloxane and decamethylcyclopenta-siloxane), which are referred to in accordance with INCI also as cyclomethicones, amino-modified silicones (INCI: Amodimethicones) and silicone waxes, e.g. polysiloxane-poly-alkylene copolymers (INCI: Stearyl Dimethicone and Cetyl Dimethicone) and dialkoxydi-methylpolysiloxanes (stearoxy dimethicone and behenoxy stearyl dimethicone), which are available as various Abil-Wax grades from Th. Goldschmidt.

The silicone oils listed below are also particularly advantageous in the sense of the present invention:

| | | | Polarity |
|---|---|---|---|
| Manufacturer | Trade name | INCI name | [mN/m] |
| Wacker | Wacker Silicone oil | Polydimethylsiloxane | 26.9 |
| | AK 100 | | |
| Wacker | Wacker Silicone oil | Polydimethylsiloxane | 46.5 |
| | AK 50 | | |
| Wacker | Wacker Silicone oil | Polydimethylsiloxane | 42.4 |
| | AK 35 | | |
| Wacker | Wacker Silicone oil | Polydimethylsiloxane | 40.9 |
| | AK 20 | | |
| Dow Corning | Dow Corning Fluid | Cyclopentasiloxane | 32.3 |
| | 245 | | |
| Dow Corning | Dow Corning Fluid | Cyclomethicone | 28.5 |
| | 345 | | |

Cyclic silicones to be used advantageously according to the invention are generally characterized by structural elements, as follows 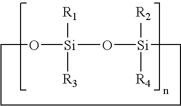
where the silicon atoms can be substituted by identical or different alkyl radicals and/or aryl radicals, which are represented here in general terms by the radicals R₁-R₄ (that is to say that the number of different radicals is not necessarily limited to 4). n can assume values from 3/2 to 20. Fractions for n take into consideration that uneven numbers of siloxyl groups may be present in the ring.

Particularly advantageous cyclic silicone oils in the sense of the present invention are cyclomethicones, in particular cyclomethicones D5 and/or cyclomethicones D6.

Advantageous silicone oils or silicone waxes in the sense of the present invention are cyclic and/or linear silicone oils and silicone waxes.

It is particularly advantageous in the sense of the present invention to choose the ratio of lipids to silicone oils to be about 1:1 (generally x:y).

Phenyltrimethicone is advantageously chosen as silicone oil. Other silicone oils, for example dimethicone, phenyldimethicone, cyclomethicone (octamethylcyclotetrasiloxane) for example hexamethylcyclotrisiloxane, polydimethylsiloxane, poly(methylphenylsiloxane), cetyldimethicone, behenoxydimethicone are also to be used advantageously in the sense of the present invention.

Also advantageous are mixtures of cyclomethicone and isotridecyl isononanoate, and those of cyclomethicone and 2-ethylhexyl isostearate.

It is, however, also advantageous to choose silicone oils of similar constitution to the above-described compounds whose organic side chains are derivatized, for example polyethoxylated and/or polypropoxylated. These include, for example, polysiloxane-polyalkyl-polyether copolymers, such as cetyl dimethicone copolyol, and cetyl dimethicone copolyol (and) polyglyceryl-4 isostearate (and) hexyl laurate.

Moreover, the preparations can comprise light filters, dyes, active ingredients, moisturizers, powder raw materials, fillers such as talc, silica, boron nitride and starch derivatives, preservatives and/or deodorants.

It is therefore advantageous in the sense of the present invention to create cosmetic and dermatological preparations whose main purpose is not protection against sunlight, but which nevertheless contain a content of UV protection substances. Thus, for example, UV-A and/or UV-B filter substances are usually incorporated into day creams or makeup products. UV protection substances, like antioxidants and, if desired, preservatives, also represent effective protection of the preparations themselves against spoilage. Also favourable are cosmetic and dermatological preparations which are present in the form of a sunscreen agent.

Accordingly, the preparations in the sense of the present invention preferably contain at least one UV-A and/or UV-B filter substance. The formulations may, but do not necessarily, optionally also contain one or more organic and/or inorganic pigments as UV filter substances, which may be present in the water phase and/or the oil phase.

Preferred inorganic photoprotective filter pigments are metal oxides and/or other metal compounds which are sparingly soluble or insoluble in water, in particular oxides of titanium (TiO₂), zinc (ZnO), iron (e.g. Fe₂O₃), zirconium (ZrO₂), silicon (SiO₂), manganese (e.g. MnO), aluminium (Al₂O₃), cerium (e.g. Ce₂O₃), mixed oxides of the corresponding metals, and mixtures of such oxides, and the sulphate of barium (BaSO₄).

The titanium dioxide pigments may be present either in the crystal modification rutile, or else in the form of anatase and may, in the sense of the present invention, be advantageously surface-treated (“coated”), the intention being to form or retain, for example, a hydrophilic, amphiphilic or hydrophobic character. This surface treatment can involve providing the pigments with a thin hydrophilic and/or hydrophobic inorganic and/or organic layer by processes known per se. The various surface coatings can also comprise water in the sense of the present invention.

Described coated and uncoated titanium dioxides can also be used in the sense of the present invention in the form of commercially available oily or aqueous predispersions.

Dispersion auxiliaries and/or solubilization promoters may advantageously be added to these predispersions.

The titanium dioxides according to the invention are characterized by a primary particle size between 10 nm to 150 nm.

| | | Additional | |
|---|---|---|---|
| | | constituents | |
| | | of the | |
| Trade name | Coating | predispersion | Manufacturer |
| MT-100TV | Aluminium hydroxide | — | Tayca |
| | stearic acid | | Corporation |
| MT-100Z | Aluminium hydroxide | — | Tayca |
| | stearic acid | | Corporation |
| MT-100F | Stearic acid | — | Tayca |
| | iron oxide | | Corporation |
| MT-500SAS | Alumina, silica | — | Tayca |
| | silicone | | Corporation |
| MT-100AQ | Silica | — | Tayca |
| | aluminium hydroxide | | Corporation |
| | alginic acid | | |
| Eusolex T-2000 | Alumina | — | Merck KGaA |
| | simethicones | | |
| Eusolex TS | Alumina, stearic acid | — | Merck KGaA |
| Titanium dioxide | None | — | Degussa |
| P25 | | | |
| Titanium dioxide | Octyltrimethylsilane | — | Degussa |
| T805 | | | |
| (Uvinul TiO₂) | | | |
| UV-Titan X170 | Alumina | — | Kemira |
| | dimethicones | | |
| UV-Titan X161 | Alumina, silica | — | Kemira |
| | stearic acid | | |
| Tioveil AQ 10PG | Alumina | Water | Solaveil |
| | silica | propylene | Uniquema |
| | | glycol | |
| Mirasun TiW 60 | Alumina | Water | Rhone-Poulenc |
| | silica | | |

In the sense of the present invention, particularly preferred titanium dioxides are MT-100Z and MT-100TV from Tayca Corporation, Eusolex T-2000 and Eusolex TS from Merck and titanium dioxide T805 from Degussa.

In the sense of the present invention, zinc oxides can also be used in the form of commercially available oily or aqueous predispersions. Zinc oxide particles suitable according to the invention and predispersions of zinc oxide particles are characterized by a primary particle size of <300 nm and are available under the following trade names from the companies listed:

| Trade name | Coating | Manufacturer |
|---|---|---|
| Z-Cote HP1 | 2% Dimethicones | BASF |
| Z-Cote | / | BASF |
| ZnO NDM | 5% Dimethicones | H&R |
| MZ 707M | 7% Dimethicones | M. Tayca Corp. |
| Nanox 500 | / | Elementis |
| ZnO Neutral | / | H&R |

Particularly preferred zinc oxides in the sense of the invention are Z-Cote HP1 from BASF and zinc oxide NDM from Haarmann & Reimer.

The total amount of one or more inorganic pigments in the finished cosmetic preparation is advantageously chosen from the range 0.1% by weight to 25% by weight, preferably 0.5% by weight to 18% by weight.

An advantageous organic pigment in the sense of the present invention is 2,2′-methylenebis(6-(2H-benzotriazol-2-yl)-4-(1,1,3,3-tetramethylbutyl)phenol) [INCI: Bisoctyltriazole], which is characterized by the chemical structural formula 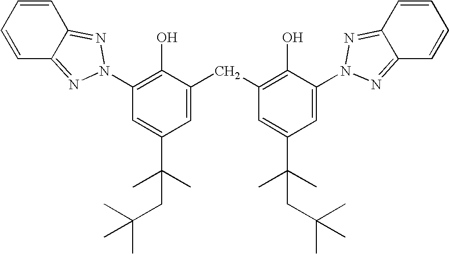
and is available under the trade name Tinosorb® M from CIBA-Chemikalien GmbH.

Advantageous UV-A filter substances in the sense of the present invention are dibenzoylmethane derivatives, in particular 4-(tert-butyl)-4′-methoxydibenzoylmethane (CAS No. 70356-09-1), which is sold by Givaudan under the name Parsole® 1789 and by Merck under the trade name Eusolex® 9020.

Further advantageous UV-A filter substances are phenylene-1,4-bis(2-benzimidazyl)-3,3′-5,5′-tetrasulphonic acid 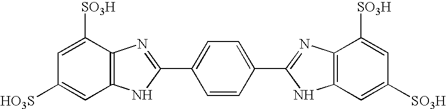
and its salts, particularly the corresponding sodium, potassium or triethanolammonium salts, in particular phenylene-1,4-bis(2-benzimidazyl)-3,3′-5,5′-tetrasulphonic bis-sodium salt 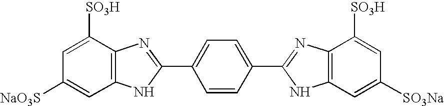
with the INCI name Bisimidazylate, which is available, for example, under the trade name Neo Heliopan AP from Haarmann & Reimer.

Also advantageous are 1,4-di(2-oxo-10-sulpho-3-bornylidenemethyl)benzene and salts thereof (in particular the corresponding 10-sulphato compounds, in particular the corresponding sodium, potassium or triethanolammonium salt), which is also referred to as benzene-1,4-di(2-oxo-3-bornylidenemethyl-10-sulphonic acid) and is characterized by the following structure:

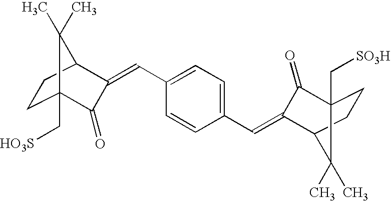

Further advantageous UV-A filter substances are hydroxybenzophenones which are characterized by the following structural formula: 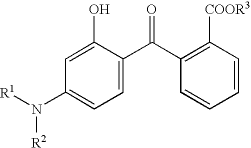
in which
R¹ and R², independently of one another, are hydrogen, C₁-C₂₀-alkyl, C₃-C₁₀-cycloalkyl or C₃-C₁₀-cycloalkenyl, where the substituents R¹ and R², together with the nitrogen atom to which they are bonded, can form a 5-membered or 6-membered ring and
R³ is a C₁-C₂₀-alkyl radical.

A particularly advantageous hydroxybenzophenone in the sense of the present invention is hexyl 2-(4′-diethylamino-2′-hydroxybenzoyl)benzoate (also: aminobenzophenone), which is characterized by the following structure: 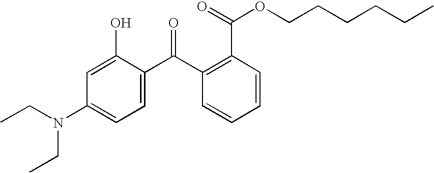
and is available under the trade name Uvinul A Plus from BASF.

Advantageous UV filter substances in the sense of the present invention are also so-called broadband filters, i.e. filter substances which absorb both UV-A and also UV-B radiation.

Advantageous broadband filters or UV-B filter substances are, for example, bisresorcinyltriazine derivatives having the following structure: 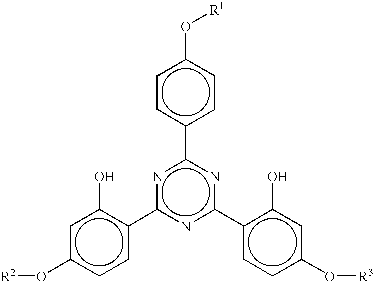
where R¹, R² and R³, independently of one another, are chosen from the group of branched and unbranched alkyl groups having 1 to 10 carbon atoms, or are a single hydrogen atom. Particular preference is given to 2,4-bis{[4-(2-ethylhexyloxy)-2-hydroxy]-phenyl}-6-(4-methoxyphenyl)-1,3,5-triazine (INCI: Aniso Triazine), which is available under the trade name Tinosorb® S from CIBA-Chemikalien GmbH.

In the sense of the present invention, particularly advantageous preparations which are characterized by high or very high UV-A protection preferably contain two or more UV-A and/or broadband filters, in particular dibenzoylmethane derivatives [for example 4-(tert-butyl)-4′-methoxydibenzoylmethane], benzotriazole derivatives [for example 2,2′-methylenebis(6-(2H-benzotriazol-2-yl)-4-(1,1,3,3-tetramethylbutyl)phenol)], phenylene-1,4-bis(2-benzimidazyl)-3,3′-5,5′-tetrasulphonic acid and/or its salts, 1,4-di(2-oxo-10-sulpho-3-bornylidenemethyl)benzene and/or salts thereof and/or 2,4-bis{[4-(2-ethylhexyloxy)-2-hydroxy]phenyl}-6-(4-methoxyphenyl)-1,3,5-triazine, in each case individually or in any combinations with one another.

Other UV filter substances, which have the structural formula 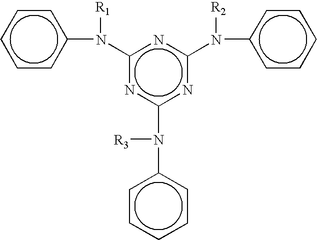
are also advantageous UV filter substances in the sense of the present invention, for example the s-triazine derivatives described in European laid-open specification EP 570 838 A1, whose chemical structure is expressed by the generic formula 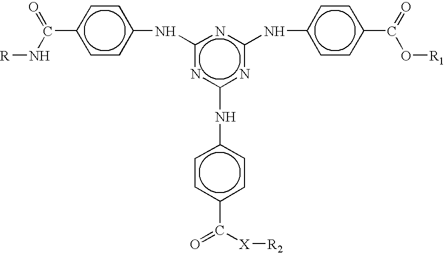
where
R is a branched or unbranched C₁-C₁₈-alkyl radical, a C₅-C₁₂-cycloalkyl radical, optionally substituted by one or more C₁-C₄-alkyl groups,
X is an oxygen atom or an NH group,
R₁ is a branched or unbranched C₁-C₁₈-alkyl radical, a C₅-C₁₂-cycloalkyl radical, optionally substituted by one or more C₁-C₄-alkyl groups, or a hydrogen atom, an alkali metal atom, an ammonium group or a group of the formula 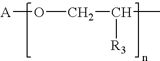
in which
   A is a branched or unbranched C₁-C₁₈-alkyl radical, a C₅-C₁₂-cycloalkyl or aryl radical, optionally substituted by one or more C₁-C₄-alkyl groups,
   R₃ is a hydrogen atom or a methyl group,
   n is a number from 1 to 10,
R₂ is a branched or unbranched C₁-C₁₈-alkyl radical, a C₅-C₁₂-cycloalkyl radical, optionally substituted by one or more C₁-C₄-alkyl groups, when X is the NH group, and
a branched or unbranched C₁-C₁₈-alkyl radical, a C₅-C₁₂-cycloalkyl radical, optionally substituted by one or more C₁-C₄-alkyl groups, or a hydrogen atom, an alkali metal atom, an ammonium group or a group of the formula 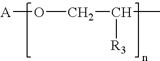
in which
   A is a branched or unbranched C₁-C₁₈-alkyl radical, a C₅-C₁₂-cycloalkyl or aryl radical, optionally substituted by one or more C₁-C₄-alkyl groups,
   R₃ is a hydrogen atom or a methyl group,
   n is a number from 1 to 10,
   when X is an oxygen atom.

A particularly preferred UV filter substance in the sense of the present invention is also an unsymmetrically substituted s-triazine, the chemical structure of which is expressed by the formula 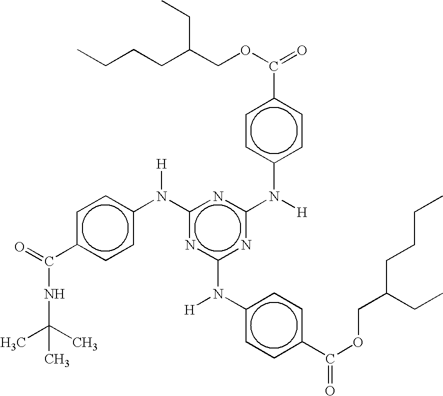
and which is also referred to below as dioctylbutylamidotriazone (INCI: Dioctylbut-amidotriazone), and is available under the trade name UVASORB HEB from Sigma 3V.

Also advantageous in the sense of the present invention is a symmetrically substituted s-triazine, tris(2-ethylhexyl) 4,4′,4″-(1,3,5-triazine-2,4,6-triyltriimino)trisbenzoate, synonym: 2,4,6-tris[anilino-(p-carbo-2′-ethyl-1′-hexyloxy)]-1,3,5-triazine (INCI: Octyl Triazone), which is marketed by BASF Aktiengesellschaft under the trade name UVINUL® T 150.

European laid-open specification 775 698 also describes bisresorcinyltriazine derivatives to be preferably used, the chemical structure of which is expressed by the generic formula 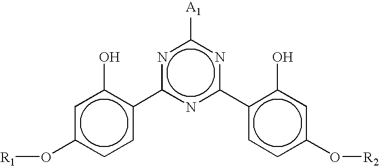
where R₁, R₂ and A₁ represent very different organic radicals.

Also advantageous in the sense of the present invention are 2,4-bis{[4-(3-sulphonato)-2-hydroxypropyloxy)-2-hydroxy]phenyl}-6-(4-methoxyphenyl)-1,3,5-triazine sodium salt, 2,4-bis{[4-(3-(2-propyloxy)-2-hydroxypropyloxy)-2-hydroxy]phenyl}-6-(4-methoxyphenyl)-1,3,5-triazine, 2,4-bis{[4-(2-ethylhexyloxy)-2-hydroxy]phenyl}-6-[4-(2-methoxyethyl-carboxyl)phenylamino]-1,3,5-triazine, 2,4-bis{[4-(3-(2-propyloxy)-2-hydroxypropyloxy)-2-hydroxy]phenyl}-6-[4-(2-ethylcarboxyl)phenylamino]-1,3,5-triazine, 2,4-bis{[4-(2-ethyl-hexyloxy)-2-hydroxy]phenyl}-6-(1-methylpyrrol-2-yl)-1,3,5-triazine, 2,4-bis{[4-tris(trimethyl-siloxysilylpropyloxy)-2-hydroxy]phenyl}-6-(4-methoxyphenyl)-1,3,5-triazine, 2,4-bis{[4-(2″-methylpropenyloxy)-2-hydroxy]phenyl}-6-(4-methoxyphenyl)-1,3,5-triazine and 2,4-bis{[4-(1′,1′,1′,3′,5′,5′,5′-heptamethylsiloxy-2″-methylpropyloxy)-2-hydroxy]phenyl}-6-(4-methoxyphenyl)-1,3,5-triazine.

An advantageous broadband filter in the sense of the present invention is 2,2′-methylenebis(6-(2H-benzotriazol-2-yl)-4-(1,1,3,3-tetramethylbutyl)phenol), which is characterized by the chemical structural formula 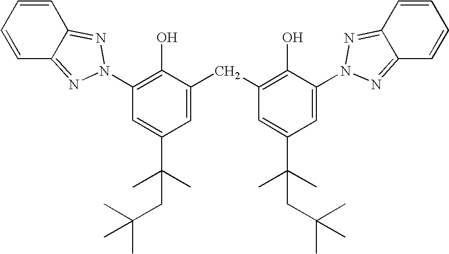
and is available under the trade name Tinosorb® M from CIBA-Chemikalien GmbH.

Another advantageous broadband filter in the sense of the present invention is 2-(2H-benzotriazol-2-yl)-4-methyl-6-[2-methyl-3-[1,3,3,3-tetramethyl-1-[(trimethylsilyl)-oxy]disiloxanyl]propyl]phenol (CAS No.: 155633-54-8) having the INCI name Drometrizole Trisiloxane, which is characterized by the chemical structural formula

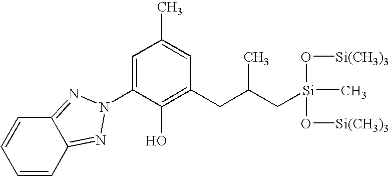

The UV-B and/or broadband filters can be oil-soluble or water-soluble. Examples of advantageous oil-soluble UV-B and/or broadband filter substances are:
3-benzylidenecamphor derivatives, preferably 3-(4-methylbenzylidene)camphor, 3-benzylidenecamphor;
4-aminobenzoic acid derivatives, preferably 2-ethylhexyl 4-(dimethylamino)benzoate, amyl 4-(dimethylamino)benzoate;
2,4,6-trianilino(p-carbo-2′-ethyl-1′-hexyloxy)-1,3,5-triazine;
esters of benzalmalonic acid, preferably di(2-ethylhexyl) 4-methoxybenzalmalonate,
esters of cinnamic acid, preferably 2-ethylhexyl 4-methoxycinnamate, isopentyl 4-methoxycinnamate;
derivatives of benzophenone, preferably 2-hydroxy-4-methoxybenzophenone, 2-hydroxy-4-methoxy-4′-methylbenzophenone, 2,2′-dihydroxy-4-methoxybenzophenone
and UV filters bonded to polymers.

Examples of advantageous water-soluble UV-B and/or broadband filter substances are:
salts of 2-phenylbenzimidazole-5-sulphonic acid, such as its sodium, potassium or its triethanolammonium salt, and also the sulphonic acid itself;
sulphonic acid derivatives of 3-benzylidenecamphor, such as, for example, 4-(2-oxo-3-bornylidenemethyl)benzenesulphonic acid, 2-methyl-5-(2-oxo-3-bornylidenemethyl)-sulphonic acid and salts thereof.

Particularly advantageous UV filter substances which are liquid at room temperature in the sense of the present invention are homomenthyl salicylate (INCI: Homosalate), 2-ethylhexyl 2-hydroxybenzoate (2-ethylhexyl salicylate, INCI: Octyl Salicylate), 4-isopropylbenzyl salicylate and esters of cinnamic acid, preferably (2-ethylhexyl) 4-methoxycinnamate (INCI: Octyl Methoxycinnamate) and isopentyl 4-methoxycinnamate (INCI: Isoamyl p-Methoxycinnamate), 3-(4-(2,2-bisethoxycarbonylvinyl)-phenoxy)propenyl)methoxysiloxane/dimethylsiloxane copolymer (INCI: Dimethicodiethyl-benzalmalonate), which is available, for example, under the trade name Parsol® SLX from Hoffmann La Roche.

A further photoprotective filter substance which can be used advantageously according to the invention is ethylhexyl 2-cyano-3,3-diphenylacrylate (octocrylene), which is available from BASF under the name Uvinul® N 539 and is characterized by the following structure:

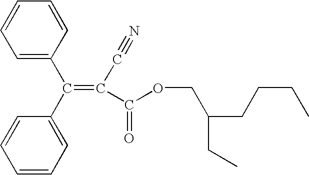

It can also be of considerable advantage to use polymer-bonded or polymeric UV filter substances in the preparations according to the present invention, in particular those described in WO-A-92/20690.

The list of specified UV filters which can be used in the sense of the present invention is not of course intended to be limiting.

The preparations according to the invention advantageously contain the substances which absorb UV radiation in the UV-A and/or UV-B region in a total amount of, for example, 0.1% by weight to 30% by weight, preferably 0.5 to 20% by weight, in particular 1.0 to 15.0% by weight, in each case based on the total weight of the preparations, in order to provide cosmetic preparations which protect the hair or the skin from the entire range of ultraviolet radiation. They can also be used as sunscreens for the hair or the skin.

Thus, as particular embodiments, the present invention relates to cosmetic and dermatological skincare and/or photoprotective preparations, in particular skincare or decorative cosmetic and dermatological photoprotective preparations with pearlescent effect.

The preparations according to the invention can advantageously, although not obligatorily, comprise preservatives.

Advantageous preservatives in the sense of the present invention are, for example, formaldehyde donors (such as, for example, DMDM hydantoin, which is available, for example, under the trade name Glydant™ from Lonza), iodopropyl butylcarbamates (e.g. those available under the trade names Glycacil-L, Glycacil-S from Lonza and/or Dekaben LMB from Jan Dekker), parabens (i.e. alkyl p-hydroxybenzoates, such as methyl-, ethyl-, propyl- and/or butylparaben), phenoxyethanol, ethanol, benzoic acid and the like. Usually, the preservative system according to the invention further advantageously also comprises preservative assistants, such as, for example, octoxyglycerol, glycine soya, etc. This list of advantageous preservatives should in no way be limiting. Instead, all preservatives approved for cosmetics or foods are advantageous in the sense of the present invention.

Moisturizers can likewise be mixed into the cosmetic preparation. Skin moisturizing agents which can be used advantageously are glycerol, chitosan, fucogel, propylene glycol, dipropylene glycol, butylene glycol, mannitol, lactic acid, sodium pyrrolidonecarboxylic acid, hyaluronic acid, salts of the given acids, and glycine, urea and salts of metals of the first and second main group.

Glycerol, lactic acid, butylene glycol, urea, hyaluronic acid are particularly suitable.

The content of skin moisturizing agents is advantageously 3% by weight to 60% by weight, preferably 4 to 50% by weight, in particular 5 to 40% by weight, based on the total weight of the preparations.

Preparations according to the invention can advantageously also comprise powders. Powders are pulverulent preparations composed of one or more powder bases which have a greater or lesser finely divided nature and to which, depending on their intended use, one or more active ingredients, preservatives, perfume oils, dyes, etc. can be added.

The FDA's OTC Miscellaneous External Panel has stipulated the following definition for powders: “A homogeneous dispersion of finely dispersed, relatively dry finely divided material which consists of one or more substances” (FDC Reports [Pink Sheet] 41, No. 33, T&G-4 [Aug. 13, 1979]).

The composition of a powder depends largely on the objectives which it has to fulfil. Powders can, however, also be diluents for medicaments, e.g. antibiotics, sulphonamides, etc. Liquid powders are mostly high-viscosity preparations (lotions) consisting of talc, zinc oxide and/or titanium dioxide, glycerol and water. Compact powders are powder bases briquetted by high pressure or caked together by adding calcium sulphate (gypsum).

Additionally, powders are also provided and used in aerosol form after it was possible to develop valves which largely exclude the possibility of the valve execution operations being obstructed.

The sedimentation of the incorporated powder particles, which is always a risk, can likewise be prevented by incorporating suitable suspending agents and/or suspension auxiliaries into the formulation, for example alkali metal, ammonium or amine salts of a dialkyl sulphosuccinate with alkyl groups of 4-12 carbon atoms, e.g. sodium dioctyl sulphosuccinate (typically about 0.002-0.015% by weight), or an alkylbenzenesulphonic acid with alkyl groups of 8-14 carbon atoms, e.g. sodium dodecylbenzenesulphonate.

Depending on the field of use, customary cosmetic fillers, additives, pigments, dyes, perfumes and also care substances and active ingredients can be incorporated into the preparations according to the invention. In formulations according to the invention it is possible to incorporate both large amounts of hydrophilic and also hydrophobic active ingredients or combinations of hydrophilic and hydrophobic active ingredients into the formulations. Such active ingredients advantageous according to the invention are, for example, acetylsalicylic acid, azulene, ascorbic acid (vitamin C), vitamin B₁, vitamin B₁₂, vitamin D₁, but also bisabolol, unsaturated fatty acids, namely the essential fatty acids (often also called vitamin F), in particular γ-linolenic acid, oleic acid, eicosapentaenoic acid, docosahexaenoic acid, camphor, extracts or other products of vegetable and animal origin, e.g. evening primrose oil, borage oil or currant seed oil, fish oils, cod liver oil or else ceramides and ceramide-like compounds and so on.

In addition, it is possible to incorporate care active ingredients which are not limited to the fat-soluble active ingredients, but can also be chosen from the group of water-soluble active ingredients, for example vitamins and the like.

A surprising property of the preparations according to the invention is that they are very good vehicles for cosmetic or dermatological active ingredients into the skin, preferred active ingredients being antioxidants which can protect the skin against oxidative stress.

It is in some cases possible and advantageous to use the preparations according to the invention as bases for pharmaceutical formulations. Corresponding requirements apply mutatis mutandis to the formulation of medicinal preparations. The transitions between pure cosmetics and pure pharmaceuticals are fluid here. Suitable pharmaceutical active ingredients according to the invention are in principle all classes of active ingredients, preference being given to lipophilic active ingredients. Examples are: antihistamines, antiphlogistics, antibiotics, antimycotics, active ingredients which promote circulation, keratolytics, antihistamines, antiphlogistics, antibiotics, antimycotics, active ingredients which promote circulation, keratolytics, hormones, steroids, vitamins, hormones, steroids, vitamins, etc.

By using amphiphilic polymers and/or associative polymers and/or siloxane elastomers (III) it is now possible for the first time to formulate emulsions with long-term stability and with cosmetic pearlescence optics which have significantly improved skin compatibility in stearate systems even when NaOH is used as the sole base for neutralization.

By virtue of the preparations according to the invention, it is additionally possible for the first time to formulate pearlescent emulsions which
have a high content of lipids, including ones of varying polarity and waxes up to a content of 30% by weight,
have long-term stability,
significantly improve the sensory properties of the hitherto very dull and sticky pearlescence systems,
have supple, soft, nonsticky, cosmetically sliding properties,
have a fatty acid content of less than 12% by weight with a hydrolysed fraction of a maximum of 9% by weight,
have significantly improved skin compatibility
can contain an increased fatty alcohol content of up to 10% by weight without impairing crystal formation and thus the pearlescence optics.

A preferred way of forming emulsions according to the invention consists in immobilizing the oil droplets through the use of hydrophobically modified, synthetic or natural polymers. Such polymers are sometimes also referred to as associative thickeners. Associative polymers include crosslinker substances, in the sense of the present description also referred to as thickeners, which form an independent gel network in which the emulsion droplets are then held by hydrophobic interaction. So-called associative and/or amphiphilic thickeners are thus then present. The network can also be held together here by the crosslinking with the emulsion droplets at the points of intersection in the network.

The invention further provides the use of the preparation as cosmetics for achieving an optically pleasing pearlescent effect. The preparations can be used and administered in the form of a cream, lotion, foams, spray. Moreover, the preparations according to the invention can be used as decorative cosmetics, makeup, whitening products, cooling products, sunscreens and, in particular, as face-, body- and hand-care products.

### EXAMPLES

| Example | 1 | 2 | 3 | 4 | 5 | 6 | 7 | 8 |
|---|---|---|---|---|---|---|---|---|
| Stearic acid/palmitic acid | 6.0 | 4.5 | 7.5 | 5.0 | 7.5 | 4.5 | 5.0 | 4.5 |
| PEG-20 stearate | | 1.5 | | | | | 0.5 | |
| PEG-40 stearate | 2.0 | | | 0.5 | 1.5 | 1.5 | | 4.5 |
| PEG-100 stearate | | | 2.5 | 1.0 | 1.0 | | 0.5 | |
| Steareth-2 | | 0.5 | | | 0.5 | | 0.6 | |
| Laureth-4 | 1.0 | | | 0.5 | | 0.6 | 0.2 | 0.5 |
| Ceteth-4 | | | 0.2 | | | 0.4 | | |
| Cetyl alcohol | 2.0 | | | 1.5 | | | 1.0 | |
| Behenyl alcohol | | | 1.5 | | | | | |
| Stearyl alcohol | | | 1.0 | | 2.5 | 0.5 | | |
| Cetearyl alcohol | | 1.5 | | | | 1.0 | | 1.5 |
| Cetyl palmitate | | | | 1.0 | | | | |
| Myristyl myristate | | 1.0 | | | | | 1.0 | |
| Dimethiconol stearate | 2.0 | | | | 4.0 | | | |
| Hydrogenated cocoglyceride | | | 1.0 | | 0.5 | | | |
| Shea butter | | 1.0 | | | | | 0.5 | |
| Silicone waxes (e.g. Abil Wax | | | 2.0 | | | | | |
| 9840) | | | | | | | | |
| Methyl palmitate | | | | 1.0 | | | 3.0 | |
| C12-15 alkyl benzoate | | | 3.0 | 3.0 | | | | |
| Butylene glycol dicaprylate/ | | | | 1.0 | | | | |
| dicaprate | | | | | | | | |
| Caprylic/capric triglyceride | | | | | | | | 1.0 |
| Ethylhexyl cocoate | | | | | | | | 2.0 |
| Octyldodecanol | | 3.0 | | | | | 3.0 | |
| Mineral oil | | 2.0 | 1.0 | | | | | |
| Hydrogenated polyisobutene | 5.0 | | | | | 4.0 | | |
| Polydecene | | 2.0 | | | 2.0 | | 1.0 | |
| Petrolatum | | | 2.0 | | 2.0 | | | |
| Cyclomethicone | 5.0 | 2.0 | 1.0 | 2.0 | | 4.0 | | 8.0 |
| Dimethicone | 1.0 | 3.0 | | 2.0 | 2.0 | 2.0 | 1.0 | 2.0 |
| Phenyltrimethicone | | | 1.0 | | | 1.0 | | |
| Dicaprylyl ether | | | 2.5 | | | | | |
| Dicaprylyl carbonate | | | | | 3.0 | | | |
| Natural oils (e.g. triglycerides | 0.5 | | | 1.0 | | | | |
| such as jojoba oil) | | | | | | | | |
| Siloxane elastomers (such as | | 1.0 | 2.0 | | 4.0 | | | |
| e.g. dimethicone/vinyl | | | | | | | | |
| dimethicone crosspolymer; | | | | | | | | |
| Dow Corning 9506 powder or | | | | | | | | |
| Polysilicone-11, Gransil | | | | | | | | |
| GCM-5) | | | | | | | | |
| Hydrophobically modified | 0.5 | 0.5 | | 0.25 | | 0.5 | | 0.5 |
| acrylates - amphiphilic | | | | | | | | |
| polymers such as e.g. | | | | | | | | |
| acrylate/alkyl acrylate | | | | | | | | |
| crosspolymer, acrylate/vinyl | | | | | | | | |
| isodecanoate crosspolymer | | | | | | | | |
| Associative polymers of the | | | 0.5 | | | 0.1 | | |
| HASE type, (such as e.g. | | | | | | | | |
| acrylate/steareth-20 | | | | | | | | |
| methacrylate copolymer, | | | | | | | | |
| acrylate/steareth-20 | | | | | | | | |
| itaconate copolymer, acrylate/ | | | | | | | | |
| steareth-50 acrylate | | | | | | | | |
| copolymer, acrylate/palmeth- | | | | | | | | |
| 25 acrylate copolymer, | | | | | | | | |
| steareth-10 ally ether/ | | | | | | | | |
| acrylate copolymer) | | | | | | | | |
| Associative polymers of the | | 0.1 | 0.1 | 0.25 | 0.5 | | 0.5 | |
| hydrophobically modified | | | | | | | | |
| POE copolymer type (such as | | | | | | | | |
| e.g. PEG-120 methylglucose | | | | | | | | |
| dioleate, PEG-60 sorbitan | | | | | | | | |
| tetraoleate, PEG-150 | | | | | | | | |
| pentaerythrityl tetrastearate, | | | | | | | | |
| PEG-55 propylene glycol | | | | | | | | |
| oleate, PEG-150 distearate, | | | | | | | | |
| PEG-180/laureth-50/TMMG | | | | | | | | |
| copolymer) | | | | | | | | |
| Trisodium EDTA | 0.2 | 0.1 | | 0.05 | | 0.1 | 0.3 | |
| Iminodisuccinate | 0.1 | | 0.1 | | 0.3 | | | 0.5 |
| Phenoxyethanol | 0.3 | 0.1 | | 0.5 | 0.8 | | | 0.4 |
| Parabens | 0.6 | | 0.4 | | 0.4 | | 0.2 | |
| Hexamidine diisethionate | | 0.1 | | | 0.05 | | 0.1 | |
| Imidodiazolydynlurea | | | | | | 0.2 | | 0.2 |
| DMDM hydantoin | | | 0.2 | | | 0.1 | | |
| Iodopropynyl butylcarbamate | | | | 0.2 | | | 0.05 | |
| Alcohol denat. | 5.0 | | 2.0 | | | 10.0 | | 8.0 |
| Octoxyglycerol | | 1.0 | | | 3.0 | | 5.0 | |
| Xanthan gum | | | | | 0.1 | | | |
| Carbomer | 0.05 | | | | | 0.2 | | |
| Polyacrylamide | | | 0.2 | | | | | 0.2 |
| Cellulose ether | | | | | | | 0.1 | |
| C18-36 acid triglyceride | | | | 0.2 | | | | |
| PVP/hexadecene copolymer | | | | 0.1 | | | | |
| Tricontayl PVP | | | | | | 0.1 | | |
| Hydroxypropylcellulose | | | 0.05 | | | | | |
| Phenylbenzimidazolesulphonic | | | | | | | 2.0 | |
| acid | | | | | | | | |
| Bisethylhexyloxyphenol | | | | 1.0 | | | | |
| methoxyphenyltriazine | | | | | | | | |
| Ethylhexyltriazone | | | | 0.5 | | | | |
| Butyl methoxydibenzoylmethane | | | | 1.0 | | | | |
| Disodium phenyldibenzimidazoletetrasulphonate | | | | | | | 1.0 | |
| Titanium dioxide T 805 | | | 0.50 | | | 0.50 | 0.50 | |
| Ethylhexyl methoxycinnamate | | | | 2.0 | | | 4.0 | |
| Octocrylene | | | 1.5 | | | | | |
| Benzophenone-3 | | | 1.5 | | | | | |
| Fillers (distarch phosphate, | | 2.0 | 4.0 | | | 3.0 | | |
| tapioca starch, aluminium | | | | | | | | |
| starch octenyl succinate, | | | | | | | | |
| silica, talc, boron nitride) | | | | | | | | |
| Perfume | q.s. | q.s. | q.s. | q.s. | q.s. | q.s. | q.s. | q.s. |
| Sodium and/or potassium | q.s. | q.s. | q.s. | q.s. | q.s. | q.s. | q.s. | q.s. |
| hydroxide solution | | | | | | | | |
| Aqua | ad. | ad. | ad. | ad. | ad. | ad. | ad. | ad. |
| | 100 | 100 | 100 | 100 | 100 | 100 | 100 | 100 |
| Examples | 9 | 10 | 11 | 12 | 13 | 14 | 15 | 16 |
| Stearic acid/palmitic acid | 10.0 | 10.0 | 7.5 | 6.0 | 10.0 | 8.5 | 10.0 | 10.0 |
| Cetyl alcohol | 0.5 | | | | | | 0.5 | |
| Behenyl alcohol | | | 0.5 | | | | | |
| Stearyl alcohol | | | | | 0.25 | | | |
| Cetearyl alcohol | | 0.25 | | | | | | 0.5 |
| Cetyl palmitate | | | | 1.0 | | | | |
| Myristyl myristate | | 1.0 | | | | | | |
| Dimethiconol stearate | 1.0 | | | | | | | |
| Hydrogenated cocoglyceride | | | 1.0 | | 0.5 | | | |
| Shea butter | | 1.0 | | | | | 0.5 | |
| Silicone waxes (e.g. Abil | | | 2.0 | | | | | |
| Wax 9840) | | | | | | | | |
| Methyl palmitate | 5.0 | 10.0 | 20.0 | 5.0 | 5.0 | 10.0 | 10.0 | 5.0 |
| C12-15 alkyl benzoate | | | 3.0 | 3.0 | | | | |
| Butylene glycol dicaprylate/ | | | | 1.0 | | | | |
| dicaprate | | | | | | | | |
| Caprylic/capric triglyceride | | | | | | | | 1.0 |
| Ethylhexyl cocoate | | | | | | | | 2.0 |
| Octyldodecanol | | 3.0 | | | | | 3.0 | |
| Mineral oil | | | 1.0 | | | | | |
| Hydrogenated polyisobutene | | | | | | 2.5 | | |
| Polydecene | | | | | 2.0 | | 1.0 | |
| Petrolatum | | | 2.0 | | 2.0 | | | |
| Cyclomethicone | 5.0 | 2.0 | 1.0 | 2.0 | | 4.0 | 5.0 | 8.0 |
| Dimethicone | 1.0 | 3.0 | | 2.0 | 5.0 | 2.0 | 1.0 | 2.0 |
| Phenyltrimethicone | | | 1.0 | | | 1.0 | | |
| Dicaprylyl ether | | | 1.0 | | | | | |
| Dicaprylyl carbonate | | | | | 3.0 | | | |
| Natural oils (e.g. triglycerides | 0.5 | | | 1.0 | | | | |
| such as jojoba oil) | | | | | | | | |
| Siloxane elastomers (such | | | 2.0 | | 4.0 | | | |
| as e.g. dimethicone/vinyl | | | | | | | | |
| dimethicone crosspolymer; | | | | | | | | |
| Dow Corning 9506 powder or | | | | | | | | |
| Polysilicone-11, Gransil | | | | | | | | |
| GCM-5) | | | | | | | | |
| Hydrophobically modified | | | | | | 0.1 | 0.1 | |
| acrylates - amphiphilic | | | | | | | | |
| polymers such as e.g. | | | | | | | | |
| acrylate/alkyl acrylate | | | | | | | | |
| crosspolymer, acrylate/vinyl | | | | | | | | |
| isodecanoate crosspolymer | | | | | | | | |
| Associative polymers of the | 0.5 | 0.5 | 0.5 | 0.25 | | 0.5 | | 0.5 |
| HASE type (such as e.g. | | | | | | | | |
| acrylate/steareth-20 | | | | | | | | |
| methacrylate copolymer, | | | | | | | | |
| acrylate/steareth-20 | | | | | | | | |
| itaconate copolymer, | | | | | | | | |
| acrylate/steareth-50 acrylate | | | | | | | | |
| copolymer, acrylate/ | | | | | | | | |
| palmeth-25 acrylate | | | | | | | | |
| copolymer, steareth-10 ally | | | | | | | | |
| ether/acrylate copolymer) | | | | | | | | |
| Associative polymers of the | | 0.1 | 0.1 | 0.25 | 0.5 | | 0.5 | |
| hydrophobically modified | | | | | | | | |
| POE copolymer type (such | | | | | | | | |
| as e.g. PEG-120 methylglucose | | | | | | | | |
| dioleate, PEG-60 | | | | | | | | |
| sorbitan tetraoleate, PEG- | | | | | | | | |
| 150 pentaerythrityl | | | | | | | | |
| tetrastearate, PEG-55 | | | | | | | | |
| propylene glycol oleate, | | | | | | | | |
| PEG-150 distearate, PEG- | | | | | | | | |
| 180/laureth-50/TMMG | | | | | | | | |
| copolymer) | | | | | | | | |
| Trisodium EDTA | 0.2 | 0.1 | | 0.05 | | 0.1 | 0.3 | |
| Iminodisuccinate | 0.1 | | 0.1 | | 0.3 | | | 0.5 |
| Phenoxyethanol | 0.3 | 0.1 | | 0.5 | 0.8 | | | 0.4 |
| Parabens | 0.6 | | 0.4 | | 0.4 | | 0.2 | |
| Hexamidine diisethionate | | 0.1 | | | 0.05 | | 0.1 | |
| Imidodiazolydynlurea | | | | | | 0.2 | | 0.2 |
| DMDM hydantoin | | | 0.2 | | | 0.1 | | |
| Iodopropynyl butylcarbamate | | | | 0.2 | | | 0.05 | |
| Alcohol denat. | 5.0 | | 2.0 | | | 10.0 | | 8.0 |
| Octoxyglycerol | | 1.0 | | | 3.0 | | 5.0 | |
| Xanthan gum | | | | | 0.1 | | | |
| Carbomer | 0.05 | | | | | 0.2 | | |
| Polyacrylamide | | | 0.2 | | | | | 0.2 |
| Cellulose ether | | | | | | | 0.1 | |
| C18-36 acid triglyceride | | | | 0.2 | | | | |
| PVP/hexadecene copolymer | | | | 0.1 | | | | |
| Tricontayl PVP | | | | | | 0.1 | | |
| Hydroxypropylcellulose | | | 0.05 | | | | | |
| Phenylbenzimidazolesulphonic | | | | | | | 1.0 | |
| acid | | | | | | | | |
| Bisethylhexyloxyphenol | | | | 1.0 | | | | |
| methoxyphenyltriazine | | | | | | | | |
| Ethylhexyltriazone | | | | 0.5 | | | | |
| Butyl methoxydibenzoylmethane | | | | 1.0 | | | | |
| Disodium phenyldibenzimidazoletetrasulphonate | | | | | | | 1.0 | |
| Titanium dioxide T 805 | | | 1.0 | | | 0.50 | 1.0 | |
| Ethylhexyl methoxycinnamate | | | | 2.0 | | | 4.0 | |
| Octocrylene | | | 2.0 | | | | | |
| Benzophenone-3 | | | 2.0 | | | | | |
| Fillers (distarch phosphate, | | 2.0 | 4.0 | | | 3.0 | | |
| tapioca starch, aluminium | | | | | | | | |
| starch octenyl succinate, | | | | | | | | |
| silica, talc, boron nitride) | | | | | | | | |
| Perfume | q.s. | q.s. | q.s. | q.s. | q.s. | q.s. | q.s. | q.s. |
| Sodium and/or potassium | q.s. | q.s. | q.s. | q.s. | q.s. | q.s. | q.s. | q.s. |
| hydroxide solution | | | | | | | | |
| Water | ad. | ad. | ad. | ad. | ad. | ad. | ad. | ad. |
| | 100 | 100 | 100 | 100 | 100 | 100 | 100 | 100 |

All of the example formulations listed produce an extraordinarily stable emulsion with pearlescence optics. In contrast to the pearlescent emulsions of the prior art, they are characterized by very good skincare properties, good skin compatibility and sensorially balanced cosmetic properties.

1-18. (canceled).

19. A cosmetic or dermatological composition which comprises:
(I) up to 10% by weight, based on a total weight of the composition, of one or more C₁₂-C₄₀ fatty acids,
(II) from 0.1% to 10% by weight, based on a total weight of the composition, of one or more C₁₂-C₄₀ fatty alcohols,
(III) from 0.01% to 10% by weight, based on a total weight of the composition, of at least one of an amphiphilic polymer, an associative polymer and a siloxane elastomer,
(IV) at least one of sodium hydroxide and potassium hydroxide,
(V) from 0.1% to 10% by weight, based on a total weight of the composition, of one or more C₁₂-C₄₀ polyethoxylated fatty acid esters having a polyethoxy chain length of from 10 to 100,
(VI) optionally, at least one low molecular weight surfactant.

20. A cosmetic or dermatological composition which comprises:
(I) up to 12% by weight, based on a total weight of the composition, of one or more C₁₂-C₄₀ fatty acids,
(II) from 0% to 3% by weight, based on a total weight of the composition, of one or more C₁₂-C₄₀ fatty alcohols,
(III) from 0.01% to 10% by weight, based on a total weight of the composition, of at least one of an amphiphilic polymer, an associative polymer and a siloxane elastomer,
(IV) at least one of sodium hydroxide and potassium hydroxide.

21. The composition of claim 19, wherein (I) comprises at least one of stearic acid and palmitic acid.

22. The composition of claim 19, wherein (II) comprises at least one of myristyl alcohol, cetyl alcohol, behenyl alcohol, stearyl alcohol and cetearyl alcohol.

23. The composition of claim 19, wherein (III) comprises at least one of dimethicone/vinyl dimethicone crosspolymer, polysilicone-11, acrylate/C₁₀₋₃₀ alkyl acrylate crosspolymer, acrylate/vinyl isodecanoate crosspolymer, acrylate/steareth-20 methacrylate copolymer, acrylate/steareth-20 itaconate copolymer, acrylate/steareth-50 acrylate copolymer, acrylate/palmeth-25 acrylate copolymer, steareth-10 allyl ether/acrylate copolymer, PEG-120 methylglucose dioleate, PEG-60 sorbitan tetraoleate, PEG-150 pentaerythrityl tetrastearate, PEG-55 propylene glycol oleate, PEG-150 distearate and PEG-180/laureth-50 TMMG copolymer.

24. The composition of claim 19, wherein (IV) comprises sodium hydroxide.

25. The composition of claim 19, wherein (V) comprises at least one of PEG-30 stearate, PEG-40 stearate and PEG-100 stearate.

26. The composition of claim 25, wherein (VI) comprises at least one of steareth-2, laureth-4 and ceteth-3.

27. The composition of claim 20, wherein (I) comprises at least one of stearic acid and palmitic acid.

28. The composition of claim 20, wherein (II) comprises at least one of myristyl alcohol, cetyl alcohol, behenyl alcohol, stearyl alcohol and cetearyl alcohol.

29. The composition of claim 20, wherein (III) comprises at least one of dimethicone/vinyl dimethicone crosspolymer, polysilicone-11, acrylate/C₁₀₋₃₀ alkyl acrylate crosspolymer, acrylate/vinyl isodecanoate crosspolymer, acrylate/steareth-20 methacrylate copolymer, acrylate/steareth-20 itaconate copolymer, acrylate/steareth-50 acrylate copolymer, acrylate/palmeth-25 acrylate copolymer, steareth-10 allyl ether/acrylate copolymer, PEG-120 methylglucose dioleate, PEG-60 sorbitan tetraoleate, PEG-150 pentaerythrityl tetrastearate, PEG-55 propylene glycol oleate, PEG-150 distearate and PEG-180/laureth-50 TMMG copolymer.

30. The composition of claim 20, wherein (IV) comprises sodium hydroxide.

31. The composition of claim 19, wherein the ratio (I):(II):(V) is from 5:1:1 to 1:1:5.

32. The composition of claim 19, wherein the ratio (I):(II):(V) is from 3:1:1 to 3:1:3.

33. The composition of claim 19, wherein the ratio (I):(II):(V) is from 3:1:1 to 1:1:3.

34. The composition of claim 19, wherein the composition is substantially free of mono- and di-fatty acid esters of glycerol and glycol.

35. The composition of claim 20, wherein the composition is substantially free of mono- and di-fatty acid esters of glycerol and glycol.

36. The composition of claim 19, wherein the composition comprises from 0.1% to 10% by weight of (I).

37. The composition of claim 20, wherein the composition comprises from 0.1% to 10% by weight of (I).

38. The composition of claim 19, wherein the composition comprises from 0.1% to 5% by weight of (II).

39. The composition of claim 36, wherein the composition comprises up to 3% by weight of (II).

40. The composition of claim 20, wherein the composition comprises from 0.1% to 5% by weight of (II).

41. The composition of claim 37, wherein the composition comprises up to 3% by weight of (II).

42. The composition of claim 19, wherein the composition comprises up to 5% by weight of (V).

43. The composition of claim 19, wherein the composition comprises from 0.01% to 5% by weight of at least one of an amphiphilic polymer and an associative polymer.

44. The composition of claim 20, wherein the composition comprises from 0.01% to 5% by weight of at least one of an amphiphilic polymer and an associative polymer.

45. The composition of claim 36, wherein the composition comprises from 0.1% to 1% by weight of at least one of an amphiphilic polymer and an associative polymer.

46. The composition of claim 37, wherein the composition comprises from 0.1% to 1% by weight of at least one of an amphiphilic polymer and an associative polymer.

47. The composition of claim 19, wherein the composition comprises at least 0.5% by weight of a siloxane elastomer.

48. The composition of claim 20, wherein the composition comprises at least 0.5% by weight of a siloxane elastomer.

49. The composition of claim 19, wherein the composition comprises:
(I) up to 10% by weight of at least one of stearic acid and palmitic acid,
(II) from 0.1% to 10% by weight of at least one of cetyl alcohol, behenyl alcohol, stearyl alcohol and cetearyl alcohol,
(III) from 0.01% to 10% by weight of at least one of dimethicone/vinyl dimethicone crosspolymer, polysilicone-11, acrylate/alkyl acrylate crosspolymer, acrylate/vinyl isodecanoate crosspolymer, acrylate/steareth-20 methacrylate copolymer, acrylate/steareth-20 itaconate copolymer, acrylate/steareth-50 acrylate copolymer, acrylate/palmeth-25 acrylate copolymer, steareth-10 allyl ether/acrylate copolymer, PEG-120 methylglucose dioleate, PEG-60 sorbitan tetraoleate, PEG-150 pentaerythrityl tetrastearate, PEG-55 propylene glycol oleate, PEG-150 distearate and PEG-180/laureth-50/TMMG copolymer,
(IV) from 0.15% to 1% by weight of sodium hydroxide,
(V) up to 10% by weight of at least one of PEG-20 stearate, PEG-40 stearate and PEG-100 stearate, and
(VI) from 0% to 10% by weight of at least one of steareth-2, laureth-4 and ceteth-3.

50. The composition of claim 20, wherein the composition comprises:
(I) up to 12% by weight of at least one of stearic acid and palmitic acid,
(II) from 0% to 3% by weight of at least one of cetyl alcohol, behenyl alcohol, stearyl alcohol and cetearyl alcohol,
(III) from 0.01% to 10% by weight of at least one of dimethicone/vinyl dimethicone crosspolymer, polysilicone-11, acrylate/alkyl acrylate crosspolymer, acrylate/vinyl isodecanoate crosspolymer, acrylate/steareth-20 methacrylate copolymer, acrylate/steareth-20 itaconate copolymer, acrylate/steareth-50 acrylate copolymer, acrylate/palmeth-25 acrylate copolymer, steareth-10 allyl ether/acrylate copolymer, PEG-120 methylglucose dioleate, PEG-60 sorbitan tetraoleate, PEG-150 pentaerythrityl tetrastearate, PEG-55 propylene glycol oleate, PEG-150 distearate and PEG-180/laureth-50/TMMG copolymer,
(IV) 0.25% to 1% by weight of sodium hydroxide.

51. The composition of claim 19, wherein the composition comprises sodium hydroxide as an exclusive neutralizing agent.

52. The composition of claim 20, wherein the composition comprises sodium hydroxide as an exclusive neutralizing agent.

53. The composition of claim 19, wherein not more than 9% of the one or more fatty acids are saponified.

54. The composition of claim 20, wherein not more than 9% of the one or more fatty acids are saponified.

55. The composition of claim 19, wherein (VI) comprises laureth-4.

56. The composition of claim 19, wherein the composition further comprises up to 30% by weight of at least one of a non-polar lipid having a polarity of at least 30 mN/m, a mineral oil, a silicone oil and a wax.

57. The composition of claim 56, wherein the non-polar lipid and the wax are selected from non-polar hydrocarbons, hydrogenated polyisobutene, squalane, cyclomethicones, dimethicones, methyl palmitate and dimethiconol stearate.

58. The composition of claim 56, wherein a lipid phase of the composition comprises up to 60% by weight, based on a total weight of the lipid phase, of one or more polar lipids having a polarity of at most 30 mN/m.

59. The composition of claim 20, wherein the composition further comprises up to 30% by weight of at least one of a non-polar lipid having a polarity of at least 30 mN/m, a mineral oil, a silicone oil and a wax.

60. The composition of claim 59, wherein the non-polar lipid and the wax are selected from non-polar hydrocarbons, hydrogenated polyisobutene, squalane, cyclomethicones, dimethicones, methyl palmitate and dimethiconol stearate.

61. The composition of claim 59, wherein a lipid phase of the composition comprises up to 60% by weight, based on a total weight of the lipid phase, of one or more polar lipids having a polarity of at most 30 mN/m.

62. The composition of claim 19, wherein the composition further comprises a solubilizer.

63. The composition of claim 62, wherein the solubilizer comprises PEG-40 hydrogenated castor oil.

64. The composition of claim 20, wherein the composition further comprises a solubilizer.

65. The composition of claim 64, wherein the solubilizer comprises PEG-40 hydrogenated castor oil.

66. The composition of claim 19, wherein the composition further comprises at least one of a photoprotective filter, a moisturizer, an active ingredient, a powder raw material, a preservative, a filler and a deodorant.

67. The composition of claim 20, wherein the composition further comprises at least one of a photoprotective filter, a moisturizer, an active ingredient, a powder raw material, a preservative, a filler and a deodorant.

68. The composition of claim 19, wherein the composition further comprises ethanol in an amount of up to 30% by weight.

69. The composition of claim 20, wherein the composition further comprises ethanol in an amount of up to 30% by weight.

70. A decorative cosmetic product which comprises the composition of claim 19.

71. A decorative cosmetic product which comprises the composition of claim 20.

72. A skin care product which comprises the composition of claim 19.

73. A skin care product which comprises the composition of claim 20.

74. A photoprotective product which comprises the composition of claim 19.

75. A photoprotective product which comprises the composition of claim 20.

76. A cleansing emulsion which comprises the composition of claim 19.

77. A cleansing emulsion which comprises the composition of claim 20.
